(19) United States
(12) Patent Application Publication
(10) Pub. No.: US 2025/0179511 A1
(43) Pub. Date: Jun. 5, 2025

(54) CROSS TALK MODULATORS AND METHODS OF USE

(71) Applicant: PIONEER HI-BRED INTERNATIONAL, INC., JOHNSTON, IA (US)

(72) Inventors: SHANE E ABBITT, ANKENY, IA (US); AJITH ANAND, DAVIS, CA (US); PRIYANKA BHYRI, JOHNSTON, IA (US); NICHOLAS DOANE CHILCOAT, CLIVE, IA (US); SCOTT DIEHN, WEST DES MOINES, IA (US); JUSTIN FRERICHS, JOHNSTON, IA (US); WILLIAM JAMES GORDON-KAMM, URBANDALE, IA (US); YI JIA, ZIONSVILLE, IN (US); SUNIL KUMAR, HYDERABAD (IN); GREGORY D MAY, ANKENY, IA (US); AMITABH MOHANTY, HYDERABAD (IN); AJIT NOTT, JOHNSTON, IA (US); NAGESH SARDESAI, JOHNSTON, IA (US); LYNNE EILEEN SIMS, POLK CITY, IA (US); XINLI EMILY WU, JOHNSTON, IA (US); STEPHANE DESCHAMPS, WEST DES MOINES, IA (US)

(73) Assignee: PIONEER HI-BRED INTERNATIONAL, INC., JOHNSTON, IA (US)

(21) Appl. No.: 18/841,562

(22) PCT Filed: Feb. 23, 2023
(86) PCT No.: PCT/US2023/063146

§ 371 Date: Aug. 26, 2024

Related U.S. Application Data
(60) Provisional application No. 63/268,625, filed on Feb. 28, 2022.

(51) Int. Cl. C12N 15/82; C07K 14/415

(52) U.S. Cl. CPC C12N 15/8216; C07K 14/415

(57) ABSTRACT

Compositions and methods are provided for the improved expression and regulation of transgenes in plants, including a method of identifying gene expression gene cross-talk blocking and modulating elements, as well as the compositions of said elements. Also provided are plant cells and plants comprising or produced by the methods and compositions described herein.

### FIELD OF THE DISCLOSURE

The present disclosure relates to the field of plant molecular biology and plant genetic engineering. More specifically, it relates to novel cross talk blocker (CTB) sequences and their use to regulate gene expression in plants.

### REFERENCE TO SEQUENCE LISTING SUBMITTED ELECTRONICALLY

The official copy of the sequence listing is submitted electronically via EFS-Web as an XML formatted sequence listing with a file named 8930-WO-PCT_SEQ_LIST_ST26.XML created on Feb. 7, 2023, and having a size of 732 kilobytes and is filed concurrently with the specification. The sequence listing comprised in this XML formatted document is part of the specification and is herein incorporated by reference in its entirety.

### BACKGROUND

Transgenic commercial crops comprise one or more transgenes that confer a desired trait, and may also contain a selectable marker transgene. The structural organization of the genome and genomic insertion sites effect the efficacy of gene expression. Additionally, transgenes in molecular stacks and the regulatory elements driving their expression can influence the expression of nearby transgenes in unpredictable ways. Transcriptional interference and transcription read-through can be observed in multi-gene stacks. This affects transgene expression, and in some cases results in mis-timed gene expression. This phenomenon led to a transgenic trait development paradigm wherein large numbers of sister events are generated and subjected to phenotyping, to identify an event with the desired phenotype.

The current corn transformation method relies on the use of morphogenic genes for immature embryo transformation and leaf transformation. These methods rely on moderate to strong (viral enhancer) expression of morphogenic genes for early response. Transient expression or removal of the morphogenic gene is important for regenerating fertile plants. The use of viral enhancer in expression cassettes perturbed expression of neighboring gene resulting in either premature gene excision (transactivation) or influenced the expression of nearby transgenes (transcriptional interference). These issues in the past were mitigated by adding multiple copies of terminator sequences, with only partial success. Polynucleotide sequences that act as “insulators” or “cross-talk blockers” have been described in animals based on their ability to block enhancer-promoter interactions and/or serve as barriers against the spreading of the silencing effects of heterochromatin. To date, little is known about cross-talk blockers in plant systems.

There is a need for methods and compositions that improve the transgene expression in plants, including eliminating the potential for transgene cross-talk between transgenes in a molecular stack.

### SUMMARY

Methods and compositions are provided for the identification, testing, and use of cross-talk blocking elements (CTBs), that improve the pattern of transgene expression in plants.

In one aspect, a recombinant polynucleotide construct is provided, comprising: at least two cassettes, wherein each cassette comprises a promoter operably linked to a heterologous gene; and at least one cross-talk blocking element; wherein the cross-talk blocking element comprises a polynucleotide sharing at least 80% identity with at least 100 contiguous nucleotides of any one of SEQ ID NO: 1-267.

In one aspect, a recombinant polynucleotide construct is provided, comprising: at least two cassettes, wherein each cassette comprises a promoter operably linked to a heterologous gene; and at least one cross-talk blocking element; wherein the cross-talk blocking element comprises any one or more motif(s) as described in Table 13.

In one aspect, a recombinant polynucleotide construct is provided, comprising: at least two cassettes, wherein each cassette comprises a promoter operably linked to a heterologous gene; and at least one cross-talk blocking element; wherein the cross-talk blocking element is a Type I or Type II cross-talk blocking element.

In one aspect, a recombinant polynucleotide construct is provided, wherein the cross-talk blocking element is adjacent to one of the at least two cassettes.

In one aspect, a recombinant polynucleotide construct is provided, wherein the cross-talk blocking element is adjacent to at least two of the at least two cassettes.

In one aspect, a recombinant polynucleotide construct is provided, wherein at least one of the promoters of the at least two cassettes is constitutive.

In one aspect, a recombinant polynucleotide construct is provided, wherein at least one of the promoters of the at least two cassettes is tissue-specific or developmental stage-specific.

In one aspect, a plant cell comprising the recombinant polynucleotide construct of any of the claims is provided. In some aspects, the plant is selected from the group consisting of: maize, soybean, Arabidopsis, canola, wheat, rice, tobacco, cotton, alfalfa, sorghum, sunflower, or safflower.

In one aspect, a transgenic plant is provided, comprising the recombinant polynucleotide construct of any of the claims in at least one cell.

In one aspect, a method for identifying a cross-talk blocking sequence is provided, the method comprising: inserting a T-DNA sequence into a first gene into a plurality of Arabidopsis plants, wherein the T-DNA sequence comprises a plurality of CaMV35S enhancer sequences at the right border, assessing the expression pattern of the genes upstream and downstream of said first gene, selecting a plant comprising an upstream or downstream gene that is not upregulated, as compared to a control plant lacking the T-DNA sequence, sequencing said upstream or downstream gene and its 5′ regulatory elements, and selecting a CTB sequence upstream of the 5′ regulatory elements.

In one aspect, a method of increasing the expression of at least one transgene in a plant cell is provided, the method comprising: introducing into the plant cell the recombinant construct of any of the claims, incubating the cell under conditions that allow the expression of the transgene, and assessing the expression of said transgene; wherein the expression of said at least one transgene is decreased compared to that of a control plant comprising the transgene but lacking the cross-talk blocker.

### BRIEF DESCRIPTION OF THE DRAWINGS

The disclosure can be more fully understood from the following detailed description and the accompanying drawings and Sequence Listing, which form a part of this application.

FIG. 1A depicts a cross-talk blocker (CTB) sequence introduced into a vector between two independent DNA expression cassettes. FIG. 1B depicts a CTB placed distantly from the DNA expression cassettes.

FIG. 2 depicts an example of an expression vector with a CTB candidate.

FIG. 3 depicts a vector schematic for testing CTB elements in Arabidopsis.

FIG. 4 depicts a schematic map of expression vector used for Agrobacterium-mediated transformation of immature maize embryos. The position of the putative insulator-like candidate being tested is highlighted with a box.

FIG. 5 is a schematic map of plasmid (SEQ ID NO:142) used for transfecting maize leaf cell protoplasts for testing CTB-like activity. The CTB candidate is inserted between the CaMV35S enhancer and the CaMV35S minimal promoter driving ZS-GREEN fluorescence gene.

FIG. 6 is a schematic map of a plasmid (SEQ ID NO:143) used for transfecting a plant cell, wherein a CTB is present as a single element.

FIG. 7 is a schematic map of a plasmid (SEQ ID NO:144) used for transfecting a plant cell, wherein a CTB is present as a pair of elements.

FIG. 8 shows results from CTB testing in a pilot protoplast assay.

FIG. 9 shows results from testing potential CTB candidates identified from Arabidopsis.

FIG. 10 shows results from testing potential CTB candidates identified from the maize genome.

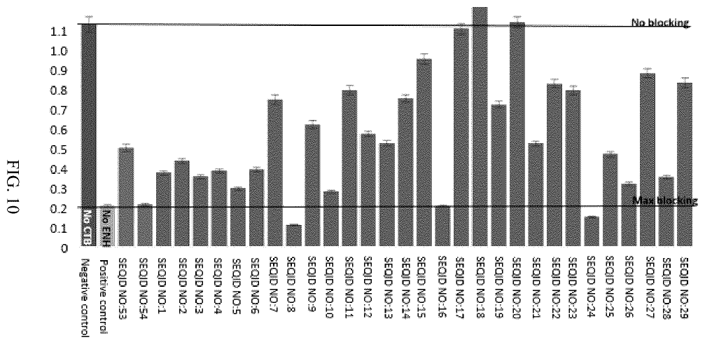

FIG. 11 depicts some of the polynucleotide motifs from CTB candidates, on the + and − strands. Numbers above sequence blocks indicate the Motif Number as listed in Table 13.

FIG. 12A-FIG. 12D depict exemplary constructs to test the hypothesis that transcriptional interference reduces the predictability of gene expression in plants. FIG. 12A represents expression of Gene 1 without influence from neighboring genes; FIG. 12B represents expression of Gene 2 without influence from neighboring genes; FIG. 12C represents transcriptional interference between two proximal genes, Gene 1 and Gene 2, in a genomic context; FIG. 12D represents a hypothetical scenario where an insulator element* (<500 bp) shields both genes from transcriptional interference. The location of insulator elements in these figures represents possible arrangements for simplicity. Other arrangements may be possible.

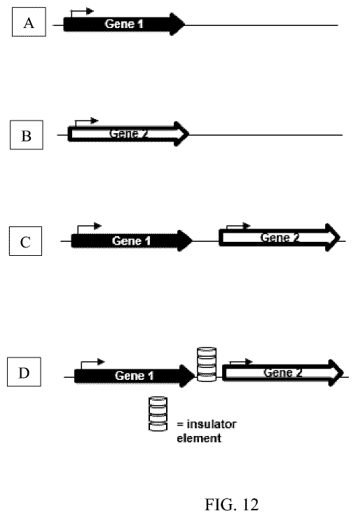

FIG. 13 is a graph that shows the relative expression patterns of vectors shown in FIG. 12A-FIG. 12D, respectively.

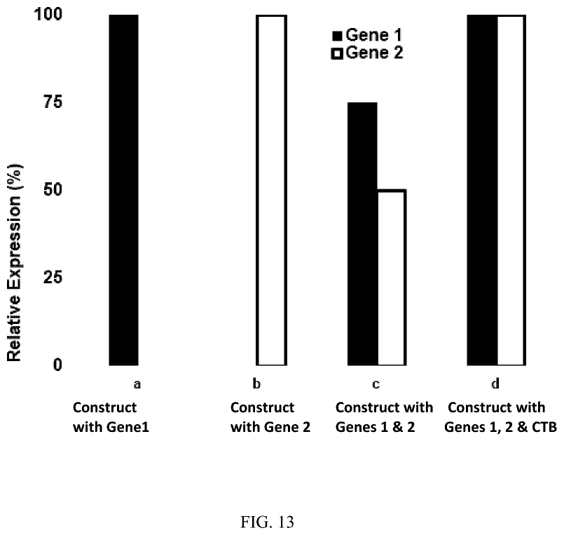

FIG. 14A-FIG. 14C depict exemplary constructs to test the hypothesis that a transcriptional enhancer reduces the predictability of gene expression in plants by influencing expression of neighboring genes. FIG. 14A represents the expression of genes in the absence of an enhancer element; FIG. 14B represents an enhancer's effects on the expression of two nearby genes; and FIG. 14C represents a hypothetical scenario where an insulator element* (<500 bp) shields a nearby gene from activation by an enhancer. The location of insulator elements in these figures represents possible arrangements for simplicity. Other arrangements may be possible.

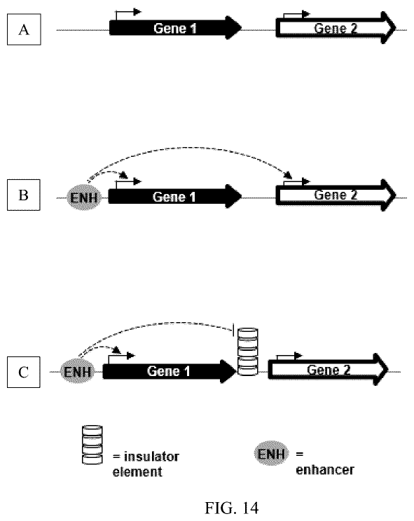

FIG. 15 is a graph that shows the relative expression patterns of vectors shown in FIG. 14A-FIG. 14C, respectively.

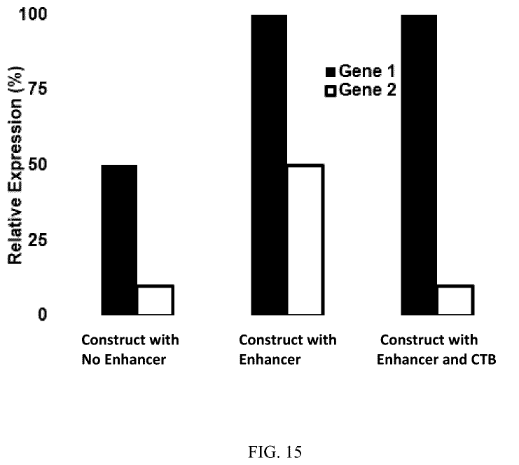

FIG. 16 depicts germline excision for marker-free SSI technology.

FIG. 17 depicts vector configurations useful in the methods disclosed herein.

### DETAILED DESCRIPTION

The structural organization of the eukaryotic genome is complex. Chromatin arrangement and the interactions between different parts of the genome as a result of chromatin structure can influence gene expression.

The ability to effectively and efficiently improve crops through genetic engineering relies on finely tuned expression of integrated genes that is predictable in varying genetic backgrounds. However, the structural organization of the eukaryotic genome is complex. The expression of a gene is not only influenced by its associated regulatory elements but may also be affected by regulatory elements of nearby genes or by transcriptional interference between genes. One strategy for improving the predictability of gene expression is to use insulator elements to shield gene expression from outside influence.

Chromatin insulators were first discovered in animals based on their ability to block enhancer-promoter interactions (enhancer blocking insulators) and/or serve as barriers against the spread of silencing effects of heterochromatin (barrier insulators). To date, little is known about insulators in plant systems.

The performance of transgenes can vary significantly in different germplasm or environments due to the interaction of transgene×genetics or transgene×genetics×environments. Thus, a thorough trait evaluation in different germplasm and environments is necessary, which increases operation cost for trait evaluation in addition to the genetics selection and improvement. One hypothesis of trait variation across germplasm and environments is due to specific regulatory elements existing in specific genetics and causing these unfavorable interactions. For example, the nearby or distal endogenous enhancers could unfavorably increase the level of transgene expression and cause the unintended agronomic consequences. On the other hand, plant genomes often contain large fraction of transposon elements which can cause unintended transgene silencing.

The issue of transcriptional interference and transcription read-through is commonly observed in multi-gene stacks. This issue affects transgene expression and in some cases results in mis-timed gene expression, which is one of the aspects that is addressed herein.

Cross Talk Blockers (CTB) or Cassette Intervening Sequences (CIS) are DNA sequences that can preserve the expression characteristics of neighboring genes in plants. The functionality of these sequences may be used for optimizing transgene expression in plants or plant cells. Their use may preserve the expression concept of a gene cassette in a context where multiple expression cassettes may be present (e.g. stacked gene configurations).

Methods and compositions of the present disclosure include a novel trait design concept and application of insulator, also known as cross talk blocker (CTB), identification and elements to improve the robustness of transgene performance across different germplasm and environments by preventing or mitigating the transgene×genetics interaction or transgene×genetics×environments interaction. Insulator is one type of regulatory elements in genome to preserve the gene expression level of their target genes by two possible modes of actions or both. One mode of action is called enhancer-blocking effect and the other is silence barrier effect. Modifications to chromatin can regulate development and response to environmental cues. Modifications can also stabilize gene expression and potentially make it more predictable.

This innovation identifies endogenous insulator elements in crop genomes and place it as part of the regulatory elements of transgenes for the traits of interest. Methods and compositions of the present disclosure further include novel plant DNA sequences that can act to block inter-cassette expression interactions in a molecular stack, and/or serve as barriers against the spreading of the silencing effects of heterochromatin. More than 800 putative insulator elements are identified by computational search and 40 insulators or insulator pairs have been identified. The validated insulator will enable the trait performance independent on the genetics and environments so that the transgenes are robust to broad germplasm and environments.

A “cross talk blocker” (CTB) is a DNA sequence of variable length (e.g., from about 15 base pairs to about 4 kb), with one or more of the following properties: a cis element upstream of a promoter, a chromatin-restructuring element (stem-loop forming sequence), a silencing barrier, an enhancer blocker, an insulator, or any combination of the preceding. When introduced, these elements potentially modulate cross-talk between different expression cassettes in a gene stack. In some embodiments, the CTB DNA sequence is about 15 base pairs to about 500 base pairs. CTB candidate sequences were characterized using multiple approaches; a) protoplast screening, b) transient and c) stable transformation. DNA sequences identified will be used to improve; a) random integration, or b) site-specific integration including recombinase-mediated and nuclease-mediated targeted integration, or c) marker-free transgenics, or d) alternate explant transformation (such as leaf or seedling-derived tissues), and/or e) cassette expression in molecular stack.

Many modifications and other aspects disclosed herein will come to mind to one skilled in the art to which the disclosed compositions and methods pertain having the benefit of the teachings presented in the following descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. Terms used in the claims and specification are defined as set forth below unless otherwise specified. It must be noted that, as used in the specification and the appended claims, the singular forms “a,” “an” and “the” include plural referents unless the context clearly dictates otherwise.

As used herein, the terms “gene expression modulating element”, “modulating element”, or “modulating sequence” refer to a polynucleotide that when it is combined with a polynucleotide of interest it does at least one of the following: a) stabilizes the polynucleotide of interest by decreasing or preventing the influence of other nearby DNA sequences b) increases the expression of the polynucleotide of interest or c) decreases the expression of the polynucleotide of interest. When referring to “gene expression modulating activity” the activity is the stabilization of, the increasing of, or the decreasing of the expression of the polynucleotide of interest. When referring to a stabilization in gene expression or an increase or decrease in gene expression, it is meant when compared to an appropriate control. For example, a control of a similar sequence size would be used to determine a gene expression modulating element. A stabilization in gene expression indicates a decrease in the variability of expression. Variability in expression of a gene of interest could be influenced by the position of the gene in the genome and/or by surrounding genes and gene elements such as enhancers, promoters, and terminators.

As used herein, the terms “gene insulator element”, “gene insulator”, “insulator”, “INS”, “CTB”, “cross-talk blocker”, “cross talk blocker”, “CIS”, “cassette intervening sequence”, or “insulator sequence” refer to a polynucleotide that, when it is combined with a polynucleotide of interest, stabilizes the polynucleotide of interest by modulating the influence of other nearby DNA sequences. Collectively, these terms are referred to as “cross-talk modulators” or “cross talk modulators”. A polynucleotide of interest includes, but is not limited to, an expression cassette comprising a promoter, gene of interest, and a terminator, or a promoter driving transcription. “Activity” with respect to these cross-talk modulators means the modification of, control of, or stabilization of the expression of a polynucleotide of interest.

The term “modulate” as used herein, refers to modifying, controlling, or stabilizing the strength of expression of a polynucleotide of interest including, but not limited to, up or down regulation.

The term “modulator” as used herein, refers to a polynucleotide that modifies, controls, or stabilizes the expression of a polynucleotide of interest including, but not limited to, up or down regulation of the polynucleotide of interest.

The term “operatively associated,” as used herein, refers to DNA sequences on a single DNA molecule which are associated so that the function of one is affected by the other. Thus, a transcription initiation region is operatively associated with a structural gene when it is capable of affecting the expression of that structural gene (i.e., the structural gene is under the transcriptional control of the transcription initiation region). The transcription initiation region is said to be “upstream” from the structural gene, which is in turn said to be “downstream” from the transcription initiation region.

“Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame.

“Intergenic region” or “intergenic sequence” is a group of nucleotides that lie in tandem and is in between two coding regions. The intergenic region is not translated.

A “cassette” is a group of nucleotide sequences that lie in tandem. A cassette is usually integrated or exchanged as a unit. For example, a DNA cassette can be the DNA that is used in transformation. It can also be the DNA that gets integrated during recombinase-mediated integration.

“Fragment” is intended a portion of the polynucleotide or a portion of the amino acid sequence and hence protein encoded thereby. Fragments of a polynucleotide may encode protein fragments that retain the biological activity of the native protein and hence influence male fertility. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the polypeptides disclosed herein.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide.

As used herein, “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic.

The similarity or relationship between two or more polynucleotide or polypeptide sequences may be determined by sequence alignment and percent identity calculations, by any method known in the art. In a non-limiting example of a mathematical algorithm utilized for the comparison of sequences is the algorithm of Needleman and Wunsch, (1970) J. Mol. Biol. 48(3):443-453, used GAP Version 10 software to determine sequence identity or similarity using the following default parameters: % identity and % similarity for a nucleic acid sequence using GAP Weight of 50 and Length Weight of 3, and the nwsgapdna.cmpii scoring matrix (watson.nih.go.jp/-gcg/man/rundata/nwsgapdna.cmp); % identity or % similarity for an amino acid sequence using GAP weight of 8 and length weight of 2, and the BLOSUM62 scoring program. Equivalent programs may also be used. “Equivalent program” is used herein to refer to any sequence comparison program that, for any two sequences in question, generates an alignment having identical nucleotide residue matches and an identical percent sequence identity when compared to the corresponding alignment generated by GAP Version 10.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

“Plant” generically includes whole plants, plant organs, plant tissues, seeds, plant cells, seeds and progeny of the same. The plant is a monocot or dicot. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). It should be noted that a protoplast is not technically an “intact” plant cell (as naturally found with all components), as protoplasts lack a cell wall. “Plant organ” refers to plant tissue or a group of tissues that constitute a morphologically and functionally distinct part of a plant. A plant element” is synonymous to a portion” of a plant, and refers to any part of the plant, and can include distinct tissues and/or organs, and may be used interchangeably with tissue” throughout. Similarly, a plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Control” or “control plant” or “control plant cell” refers to a reference for measuring changes in phenotype of the subject organism or cell.

“Somatic embryo” is defined as a multicellular structure that progresses through developmental stages that are similar to the development of a zygotic embryo, including formation of globular and transition-stage embryos, formation of an embryo axis and a scutellum, and accumulation of lipids and starch. Single somatic embryos derived from a zygotic embryo germinate to produce single non-chimeric plants, which may originally derive from a single-cell.

Embryogenic callus is defined as a friable or non-friable mixture of undifferentiated or partially undifferentiated cells which subtend proliferating primary and secondary somatic embryos capable of regenerating into mature fertile plants.

Somatic meristem is defined as a multicellular structure that is similar to the apical meristem which is part of a seed-derived embryo, characterized as having an undifferentiated apical dome flanked by leaf primorida and subtended by vascular initials, the apical dome giving rise to an above-ground vegetative plant. Such somatic meristems can form single or fused clusters of meristems.

Organogenic callus is defined as a compact mixture of differentiated growing plant structures, including but not limited to apical meristems, root meristems, leaves and roots.

Germination is the growth of a regenerable structure to form a plantlet which continues growing to produce a plant.

“Trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance

“Polynucleotide of interest” includes any nucleotide sequence encoding a protein or polypeptide that improves desirability of crops, i.e. a trait of agronomic interest. Polynucleotides of interest include, but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

“Expression cassette” as used herein means a DNA construct comprising a regulatory element of the embodiments operably linked to a heterologous polynucleotide expressing a transcript or gene of interest. Such expression cassettes will comprise a transcriptional initiation region comprising one of the regulatory element polynucleotide sequences of the present disclosure, or variants or fragments thereof, operably linked to the heterologous nucleotide sequence. Such an expression cassette may be provided with a plurality of restriction sites for insertion of the polynucleotide sequence to be under the transcriptional regulation of the regulatory regions. The expression cassette may additionally contain selectable marker genes as well as 3′ termination regions

“Promoter” is a region of DNA involved in recognition and binding of RNA polymerase and other proteins to initiate transcription. The promoter sequence consists of proximal and more distal upstream elements, the latter elements often referred to as enhancers. “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters useful for marker free CRE-mediated excision include those expressed in reproductive tissues or cells including, but not limited to, ear, tassel, ovule, anther, and more particularly germline cells such as egg, pollen, or sperm.

### Recombinant Constructs for Plant Transformation

The compositions disclosed herein, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in traits of agronomic interest such as but not limited to, crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like. In further embodiments, genes encoding pesticidal proteins may include insecticidal proteins from Pseudomonas sp. such as PSEEN3174 (Monalysin, (2011) PLoS Pathogens, 7:1-13), from Pseudomonas protegens strain CHAO and Pf5 (previously fluorescens) (Pechy-Tarr, (2008) Environmental Microbiology 10:2368-2386: GenBank Accession No. EU400157); from Pseudomonas taiwanensis (Liu, et al., (2010) J. Agric. Food Chem. 58:12343-12349) and from Pseudomonas pseudoalcaligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of U.S. Pat. No. 9,688,730; an AfIP-1A and/or AfIP-1B polypeptide of U.S. Pat. No. 9,475,847; a PIP-47 polypeptide of U.S. Pat. No. 10,006,045; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of International Patent Application Publication Number WO2018/075350; an IPD078 polypeptide, an IPD084 polypeptide, an IPD085 polypeptide, an IPD086 polypeptide, an IPD087 polypeptide, an IPD088 polypeptide, and an IPD089 polypeptide of International Patent Application Publication Number WO2018/084936; PIP-72 polypeptide of US Patent Publication Number US20160366891; a PtIP-50 polypeptide and a PtIP-65 polypeptide of US Patent Application Publication Number US20170166921; an IPD098 polypeptide, an IPD059 polypeptide, an IPD108 polypeptide, an IPD109 polypeptide of International Patent Application Publication Number WO2018/232072; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of International Patent Application Publication Number WO 2017/105987, an IPD090 polypeptide of International Patent Application Publication Number WO2017/192560, an IPD093 polypeptide of International Patent Application Publication Number WO2018/111551; an IPD103 polypeptide of International Patent Application Publication Number WO2018/005411; an IPD101 polypeptide of International Patent Application Publication Number WO2018/118811; an IPD121 polypeptide of International Patent Application Publication Number WO2018/208882, and 6-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35,Cry36, Cry37, Cry38, Cry39, Cry40, Cry41, Cry42, Cry43, Cry44, Cry45, Cry 46, Cry47, Cry49, Cry50, Cry51, Cry52, Cry53, Cry 54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of 6-endotoxin genes and the B. thuringiensis cytolytic Cyt1 and Cyt2 genes.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK:sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron. Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Accl-S1, Accl-S2 and Accl-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72); Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

Methods are available in the art for synthesizing plant-preferred genes. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

A polynucleotide encoding a gene may be functionally linked to a heterologous expression element, to facilitate transcription or regulation in a host cell. Such expression elements include but are not limited to: promoter, leader, intron, and terminator.

Expression of heterologous DNA sequences in a plant host is dependent upon the presence of operably linked promoters, including promoters, that are functional within the plant host. Choice of the promoter sequence will determine when and where within the organism the heterologous DNA sequence is expressed. Where expression in specific tissues or organs is desired, tissue-preferred promoters may be used. Where gene expression in response to a stimulus is desired, inducible promoters are the regulatory element of choice. In contrast, where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

Frequently it is desirable to express a DNA sequence in particular tissues or organs of a plant. For example, use of tissue-preferred promoters operably linked to morphogenic genes that promote cell proliferation are useful for the efficient recovery of transgenic events during the transformation process. Such tissue-preferred promoters also have utility in expressing trait genes and/or pathogen-resistance proteins in the desired plant tissue to enhance plant yield and resistance to pathogens. Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that expression of the antisense sequence produces an RNA transcript that interferes with translation of the mRNA of the native DNA sequence.

Additionally, it may be desirable to express a DNA sequence in plant tissues that are in a particular growth or developmental phase such as, for example, cell division or elongation. Such a DNA sequence may be used to promote or inhibit plant growth processes, thereby affecting the growth rate or architecture of the plant.

Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013103367 published 11 Jul. 2013, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1129-38; Matsuoka et al., (1993) Proc. Natl. Acad. Sci. USA 90:9586-90; Simpson et al., (1958) EMBOJ4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes rolC and rolD root-inducing genes); Teeri et al., (1989) EMBOJ8:343-50 (Agrobacterium wound-induced TR1′ and TR2′ genes); VfENOD-GRP3 gene promoter (Kuster et al., (1995) Plant Mol Biol 29:759-72); and rolB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); and for example those disclosed in WO2000011177 published 2 Mar. 2000 and U.S. Pat. No. 6,225,529. For dicots, seed-preferred promoters include, but are not limited to, bean 0-phaseolin, napin, 0-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO2000012733 published 9 Mar. 2000, where seed-preferred promoters from END1 and END2 genes are disclosed.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO1993001294 published 21 Jan. 1993), activated by hydrophobic electrophilic compounds used as pre-emergent herbicides, and the tobacco PR-la promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Pathogen inducible promoters induced following infection by a pathogen include, but are not limited to those regulating expression of PR proteins, SAR proteins, beta-1,3-glucanase, chitinase, etc.

A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

Another example of an inducible promoter useful in plant cells, is the ZmCAS1 promoter, described in US20130312137 published 21 Nov. 2013.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, NY:Academic Press), pp. 1-82.

### Cross-Talk Modulating Elements

In addition to promoters, other non-coding elements may regulate the expression of a gene. Such elements include insulators or “cross-talk blockers” (CTBs) that block enhancer-promoter interactions and/or serve as barriers against the spreading of the silencing effects of heterochromatin.

Examples of CTB elements include SEQ ID NO: 1-267, as well as functional fragments and variants thereof. In some aspects, a functional fragment or variant comprises at least one motif characteristic of a Type I or Type II CTB. Type I CTBs are capable of enhancer-blocking activity. Type II CTBs are capable of both enhancer-blocking and silence barrier activities.

In some aspects, the CTB comprises a motif described in Table 13.

In some aspects, the CTB shares at least at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 25, between 25 and 50, at least 50, between 50 and 75, at least 75, between 75 and 100, at least 100, or greater than 100 contiguous or non-contiguous nucleotides of a sequence selected from the group consisting of SEQ ID NO: 1-267.

### Transformation

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient provision of a nucleic acid, as well as the stable transformation of a nucleic acid into a cell.

The methods of the invention involve introducing a nucleotide construct or a polypeptide into a plant. By “introducing” is intended presenting to the plant the nucleotide construct (i.e., DNA or RNA) or a polypeptide in such a manner that the nucleic acid or the polypeptide gains access to the interior of a cell of the plant. The methods of the invention do not depend on a particular method for introducing the nucleotide construct or the polypeptide to a plant, only that the nucleotide construct gains access to the interior of at least one cell of the plant. Methods for introducing nucleotide constructs and/or polypeptides into plants are known in the art including, but not limited to, stable transformation methods, transient transformation methods, virus-mediated methods, DNA integration recombinase systems.

By “stable transformation” is intended that the nucleotide construct introduced into a plant integrates into the genome of the plant and is capable of being inherited by progeny thereof. By “transient transformation” is intended that a nucleotide construct or the polypeptide introduced into a plant does not integrate into the genome of the plant.

In preparing a DNA cassette, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved. The DNA cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvi-rus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Mace-jak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods or sequences known to enhance translation can also be utilized, for example, introns, and the like.

The method of transformation is not critical to the invention; various methods of transformation are currently available. As newer methods are available to transform host cells they may be directly applied. Accordingly, a wide variety of methods have been developed to insert a DNA sequence into the genome of a host cell to obtain the transcription and/or translation of the sequence. Thus, any method that provides for efficient transformation/transfection may be employed.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

Plant cells differ from animal cells (such as human cells), fungal cells (such as yeast cells) and protoplasts, including for example plant cells comprise a plant cell wall which may act as a barrier to the delivery of components.

Protocols for introducing polynucleotides, polypeptides or polynucleotide-protein complexes into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s):Gerhardt, Rosario. Publisher:InTech, Rijeka, Croatia. CODEN:69PQBP; ISBN:978-953-307-201-2), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture:Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, New York), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Methods for transforming various host cells are disclosed in Klein et al. “Transformation of microbes, plants and animals by particle bombardment”, Bio/Technol. New York, N.Y., Nature Publishing Company, March 1992, 10(3):286-291. Techniques for transforming a wide variety of higher plant species are well known and described in the technical, scientific, and patent literature. See, for example, Weising et al, Ann. Rev. Genet. 22:421-477 (1988).

For example, the DNA construct may be introduced directly into the genomic DNA of the plant cell using techniques such as electroporation, PEG-induced transfection, particle bombardment, silicon fiber delivery, or microinjection of plant cell protoplasts or embryogenic callus. See, e.g. Tomes et al. Direct DNA Transfer into Intact Plant Cells Via Microprojectile Bombardment, pp. 197-213 in Plant Cell, Tissue and Organ Culture, Fundamental Methods, eds. O. L. Gamborg and G. C. Phillips. Springer-Verlag Berlin Heidelberg N.Y, 1995. The introduction of DNA constructs using polyethylene glycol precipitation is described in Paszkowski et al, Embo J. 3:2717-2722 (1984). Electroporation techniques are described in Fromm et al. Proc. Natl. Acad. Sci. 82:5824 (1985). Ballistic transformation techniques are described in Klein et al. Nature 327:70-73 (1987).

Alternatively, the DNA constructs may be combined with suitable T-DNA flanking regions and introduced into a Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-meditated transformation techniques are well described in the scientific literature. See, for example Horsch et al. Science 233:496-498 (1984), and Fraley et al. Proc. Natl. Acad. Sci. 80:4803 (1983). For instance, Agrobacterium transformation of maize is described in U.S. Pat. No. 5,981,840. Agrobacterium transformation of monocot is found in U.S. Pat. No. 5,591,616. Agrobacterium transformation of soybeans is described in U.S. Pat. No. 5,563,055.

Other methods of transformation include (1) Agrobacterium rhizogenes-induced transformation (see, e.g., Lichtenstein and Fuller In: Genetic Engineering, vol. 6, P W J Rigby, Ed, London, Academic Press, 1987; and Lichtenstein, C. P, and Draper, J, In: DNA Cloning, Vol. II, D. M. Glover, Ed, Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-induced DNA uptake (see, e.g. Freeman et al. Plant Cell Physiol. 25:1353, 1984), (3) the vortexing method (see, e.g. Kindle, Proc. Natl. Acad. Sci, USA 87:1228, (1990).

DNA can also be introduced into plants by direct DNA transfer into pollen as described by Zhou et al. Methods in Enzymology 101:433 (1983); D. Hess, Intern Rev. Cytol. 107:367 (1987); Luo et al. Plant Mol. Biol. Reporter, 6:165 (1988). Expression of polypeptide coding nucleic acids can be obtained by injection of the DNA into reproductive organs of a plant as described by Pena et al. Nature 325:274 (1987). Transformation can also be achieved through electroporation of foreign DNA into sperm cells then microinjecting the transformed sperm cells into isolated embryo sacs as described in U.S. Pat. No. 6,300,543 by Cass et al. DNA can also be injected directly into the cells of immature embryos and the rehydration of desiccated embryos as described by Neuhaus et al, Theor. Appl. Genet. 75:30 (1987); and Benbrook et al, in Proceedings Bio Expo 1986, Butterworth, Stoneham, Mass, pp. 27-54 (1986).

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker which has been introduced together with a polynucleotide of the present invention. For transformation and regeneration of maize see, Gordon-Kamm et al. The Plant Cell 2:603-618 (1990).

Other methods of introducing polynucleotides into a prokaryotic and eukaryotic cell or organism or plant part can be used, including plastid transformation methods, and the methods for introducing polynucleotides into tissues from seedlings or mature seeds.

### Cell Genome Modification

Compositions that have been introduced into a cell via transformation may be integrated into the genome of a cell, by any method known in the art, for example but not limited to: TALENs, CRISPR, Meganucleases, Recombinases, and the like.

Methods to modify or alter endogenous genomic DNA are known in the art. In some aspects, methods and compositions are provided for modifying naturally-occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including meganucleases (WO 2009/114321; Gao et al. (2010) Plant Journal 1:176-187), restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks, and includes homologous recombination (HR) and single-strand annealing (SSA) (Lieber. 2010 Annu. Rev. Biochem. 79:181-211). A CRISPR-Cas system comprises, at a minimum, a CRISPR RNA (crRNA) molecule and at least one CRISPR-associated (Cas) protein to form crRNA ribonucleoprotein (crRNP) effector complexes.

CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner (Jore et al., Nature Structural & Molecular Biology 18, 529-536 (2011)). The crRNA serves as a guide RNA for sequence specific binding of the effector (protein or complex) to double strand DNA sequences, by forming base pairs with the complementary DNA strand while displacing the noncomplementary strand to form a so called R-loop. (Jore et al., 2011. Nature Structural & Molecular Biology 18, 529-536).

Another example for genetically modifying the cell or plant described herein, is by using “custom” meganucleases produced to modify plant genomes (see e.g., WO 2009/114321; Gao et al. (2010) Plant Journal 1:176-187. The term “meganuclease” generally refers to a naturally-occurring homing endonuclease that binds double-stranded DNA at a recognition sequence that is greater than 12 base pairs and encompasses the corresponding intron insertion site. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease.

TAL (transcription activator-like) effectors from plant pathogenic Xanthomonas are important virulence factors that act as transcriptional activators in the plant cell nucleus, where they directly bind to DNA via a central domain of tandem repeats. A transcription activator-like (TAL) effector-DNA modifying enzymes (TALE or TALEN) are also used to engineer genetic changes. See e.g., US20110145940, Boch et al., (2009), Science 326(5959): 1509-12. Fusions of TAL effectors to the FokI nuclease provide TALENs that bind and cleave DNA at specific locations. Target specificity is determined by developing customized amino acid repeats in the TAL effectors.

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks, and includes homologous recombination (HR) and single-strand annealing (SSA) (Lieber. 2010 Annu. Rev. Biochem. 79:181-211). HR pathways may be utilized for the insertion of a transgene or other heterologous element into the genome of the cell.

Integration of a heterologous polynucleotide into the genome of a cell may also be accomplished by the use of recombinases, for the insertion of “landing pads” int the genome of the cell. Examples of recombination sites for use in the invention are known in the art and include FRT sites (See, for example, U.S. Pat. No. 6,187,994; Schlake and Bode (1994) Biochemistry 33:12746-12751; Huang et al. (1991) Nucleic Acids Research 19:443-448; Paul D. Sadowski (1995) In Progress in Nucleic Acid Research and Molecular Biology 51:53-91; Michael M. Cox (1989) In Mobile DNA, Berg and Howe (eds) American Society of Microbiology, Washington D.C, pp. 116-670; Dixon et al. (1995) 18:449-458; Umlauf and Cox (1988) The EMBO Journal 7:1845-1852; Buchholz et al. (1996) Nucleic Acids Research 24:3118-3119; Kilby et al. (1993) Trends Genet. 9:413-421; Rossant and Geagy (1995) Nat. Med. 1:592-594; Albert et al. (1995) The Plant J. 7:649-659; Bayley et al. (1992) Plant Mol. Biol. 18:353-361; Odell etal. (1990) Mol. Gen. Genet. 223:369-378; and Dale and Ow (1991) Proc. Natl. Acad. Sci. USA 88:10558-105620; all of which are herein incorporated by reference); lox (Albert et al. (1995) Plant J. 7:649-659; Qui et al. (1994) Proc. Natl. Acad. Sci. USA 91:1706-1710; Stuurman et al. (1996) Plant Mol. Biol. 32:901-913; Odell et al. (1990) Mol. Gen. Gevet. 223: 369-378; Dale etal. (1990) Gene 91:79-85; and Bayley et al. (1992) Plant Mol. Biol. 18:353-361.) Dissimilar recombination sites are designed such that integrative recombination events are favored over the excision reaction. Such dissimilar recombination sites are known in the art. For example, Albert et al. introduced nucleotide changes into the left 13 bp element (LE mutant lox site) or the right 13 bp element (RE mutant lox site) of the lox site. Recombination between the LE mutant lox site and the RE mutant lox site produces the wild-type loxP site and a LE+RE mutant site that is poorly recognized by the recombinase Cre, resulting in a stable integration event (Albert etal. (1995) Plant J. 7:649-659). See also, for example, Araki et al. (1997) Nucleic Acid Research 25:868-872.

Using any of the methods known in the art, a heterologous polynucleotide may be integrated into the genome of a cell.

A variety of methods are available to identify those cells having an altered genome, with or without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Cells and Plants

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

Examples of monocot plants that can be used include, but are not limited to: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), teff (Eragrostis species), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to: soybean (Glycine max), Brassica species (for example but not limited to: oilseed rape or Canola) (Brassica napus, Brassica campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense, Gossypium hirsutum), and peanut (Arachis hypogaea), tomato (Solanum lycopersicum), potato (Solanum tuberosum.

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Caricapapaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true first such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention.

The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

A wide range of tissue or explant types can be used in the current method, including suspension cultures, protoplasts, immature embryos, mature embryos, immature cotyledons, mature cotyledons, split seed, embryonic axes, hypocotyls, epicotyls and leaves. Methods and compositions for the transformation and regeneration of crop plants, such as but not limited to maize, soybean, wheat, alfalfa, canola, rice, sugarcane, cotton, and others are known in the art. Standard protocols for various methods for introducing components into plant cells include, but are not limited to, methods for particle bombardment (Finer and McMullen, 1991, In Vitro Cell Dev. Biol.-Plant 27:175-182), Agrobacterium-mediated transformation (Jia et al., 2015, Int J. Mol. Sci. 16:18552-18543; US20170121722), or Ochrobactrum-mediated transformation (US20180216123) for soybean, or methods for corn such as described in WO2017074547A1, can be used with the methods of the disclosure. These methods are listed as non-limiting examples.

Additional compositions, such as morphogenic factors (e.g., developmental genes, such as Babyboom and/or Wuschel) may improve the frequency of transformation. See, for example, US20170121722A1 published 4 May 2017. Other compositions, such as regulatory expression elements, may be selected for various attributes, such as but not limited to, temporal or spatial regulation of gene expression.

### Example 1: Identification of Insulator Sequences

Different searching strategies were designed to computationally identify two different types of insulators based on the expected attributes of enhancer blocking only (for type I insulator) and combination of enhancer blocking and silence barrier (for type II insulator), as described in Table 1.

TABLE 1
Type I and Type II insulators with associated attributes

Insulator Types

| Insulating effect | I | II |
|---|---|---|
| Enhancer blocking | √ | √ |
| Silence barrier | | √ |

The target genes for insulator I were selected based on the adjacent gene expression patterns (i.e. low and high expression levels between genes in pair). The open chromatin sequences interacted with these genes were used for motif enrichment. Motifs were mapped back to the targeted anchor sequences for motif cluster identification. These sequences were then used for the motif enrichment procedure for type I insulator discovery and validation. For insulator II, the targeted genes were identified by the expected stable expression pattern across different tissue types. The rest of the procedures were similar with insulator I process.

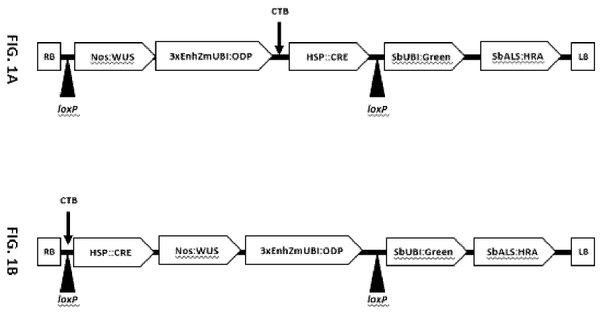

An insulator, or cross-talk blocker, was defined as a DNA sequence of variable length (˜20 bp-2 kb) which fell in any of the following category including: a cis element, a chromatin association element (stem-loop forming sequence), silencing barrier, enhancer blockers, an insulator, or any combination thereof. When introduced, these elements potentially block cross-talk between genes on a T-DNA, either tandemly arranged as two independent DNA expression cassettes (FIG. 1A) or are placed distantly (FIG. 1B) or in the physical context of chromosomal gene. These blocker elements can be placed in the 5′, 3′ or combined ends of the protected DNA expression units.

### Example 2: Expression Assay of Reporter Gene in Maize Protoplasts

Transfection vectors were built with two expression cassettes. One cassette was used for normalization to eliminate the effects of plasmid copy number variations in the protoplast population. The second cassette was used for evaluating the expression effects of each insulator candidate.

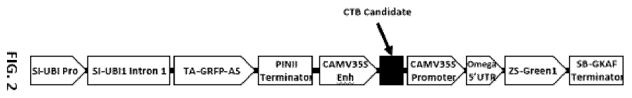

The normalization cassette (example depicted in FIG. 2) comprised a strong constitutive regulatory element (Seteria italica ubiquitin promoter and first intron) driving TagRFP with a PINII terminator (Solanum tuberosum invertase). The experimental cassette comprised the CAMV35S promoter divided into a 49 bp minimal promoter and a 433 bp upstream enhancer. The division was made at a position 16 upstream of the TATA sequence. This promoter was paired with the Omega prime 5′ untranslated region from the Tobacco Mosaic Virus. Together, these elements drove ZsGreen1 as the reporter gene with the Sorghum bicolor gamma kafarin terminator.

Insulator candidates were cloned between the CAMV35S enhancer and minimal promoter. Insulation was observed as decreased levels of fluorescence from ZS-Green1. The negative control (no insulation) was a vector with no insulator separating the CAMV35S enhancer and minimal promoter. The positive control (max insulation) was a vector with only the minimal promoter (e.g. no CAMV35S enhancer). The CAMV35S minimal promoter produced no ZS-Green1 fluorescence in the absence an enhancer.

Vectors were tested in maize leaf protoplasts using a modified version of a commonly used protocol to facilitate the delivery of known plasmid DNA to cells isolated from maize inbred leaf mesophyll cells. Transfection was achieved using 40% (w/v) polyethelene glycol for 15 minutes.

The quantification of fluorescence was performed using a Cytation5 inverted microscope imager (Biotek). Images were taken at 4× of the transfected protoplast populations using excitation and emission spectra based on the fluorescent markers. Post-imaging processing was carried using the BioTek Gen5 software. Using a circularity, size, and presence of TagRFP fluorescence algorithm, positively transfected cells were identified and the relative fluorescence, based on pixel intensity, was recorded. The fluorescence recorded from the GFP channel was normalized to the RFP in order to quantify on a cell by cell basis. The geometric mean was calculated for each experimental entity and compared to the appropriate control with 95% confidence intervals.

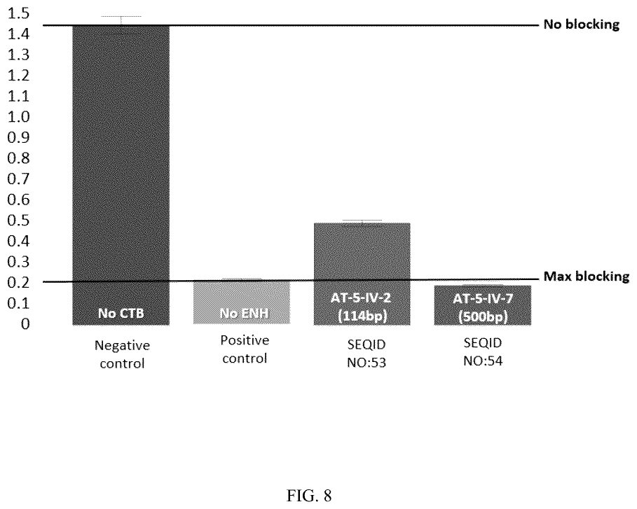

Results from the protoplast pilot study are depicted in FIG. 8.

### Example 3: Testing of Cross-Talk Blockers for Agrobacterium-Mediated Immature Embryo Site-Specific Integration (SSI)

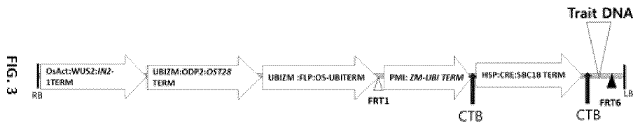

Arabidopsis CTB elements previously described in U.S. Pat. No. 7,655,786 B2 were selected. Three of the DNA fragments, 5-III-1, 5-IV-2, and 5-IV-7, were selected for testing for immature embryo marker-free SSI. DNA expression cassettes containing the above elements were designed and placed on the 3′ and/or 5′ end the cassette. A schematic design of the vector is provided in FIG. 3. The T-DNA vector is comprised of the following components: right border, the rice actin promoter, rice actin intron, driving expression of a maize WUS2 coding sequence and maize IN2-1 terminator; maize ubiquitin promoter, 5′UTR, ubiquitin intron driving the expression of a maize ODP2 coding sequence and maize OST28 terminator; maize ubiquitin promoter, 5′UTR, ubiquitin intron driving the expression of a maize optimized FLP EXON1, ST-LS 1 INTRON2 followed by maize optimized FLP EXON1 coding sequence and rice ubiquitin terminator. A DNA with the recombination site FRT1 flanking a promoter-less pmi gene encoding the phosphomannose isomerase conferring resistance to mannose with maize ubiquitin terminator. A heat shock promoter HSP17.7 driving the expression of maize optimized Cre EXON1, ST-LS 1 INTRON2 followed by a Cre EXON2 coding sequence and Sorghum bicolor C18 terminator. A trait gene cassette with viral enhancers fused to a promoter driving the expression of any trait gene followed by the recombination site FRT6. The CTB elements are placed either 3′ end of the HSP:Cre expression cassette and/or at both at 3′ and 5′ of the Cre expression cassette to insulate the HSP promoter from promoter-enhancer activation or transcriptional interference. As a consequence of the insulation, higher rates of SSI events which are free of the marker gene and HSP:CRE cassette were recovered at TO level.

The T-DNA was transformed into Agrobacterium strain LBA4404 TD Thy- and used for transforming immature embryos derived transgenic plants with recombinant target line (RTL) containing the heterologous recombination sites FRT/16 or FRT1/87. The different steps in transformation, event selection and molecular analysis of SSI events is disclosed in US20170240911A1. The events which were free of marker-gene, Cre, morphogenic genes (WUS2 and ODP2) and FLP, but have an intact copy of the trait gene and FRT6 site inserted in RTL were identified as clean SSI events. This method allowed to improve the frequency of SSI events compared to constructs without the CTB sequence for insulation. A similar vector design without the donor template is used for mitigating promoter-enhancer and transcriptional interference in random immature embryo transformation and for expressing morphogenic genes.

### Example 4: CTB Identification from Arabidopsis Activation-Tagged Lines

Activation-tagged lines in Arabidopsis (Weigel et al 2000) are T-DNA insertion lines with 4 copies of the Cauliflower Mosaic Virus (CaMV) 35S enhancer situated at the right border of the T-DNA. The insertion of the T-DNA in the genome can have several effects. Insertion of the T-DNA into a gene or its regulatory element could disrupt the expression of the gene, while insertion of the T-DNA in intergenic regions could trigger the expression of flanking or neighboring genes as a result of transactivation by the CaMV35S enhancers. In other cases, the T-DNA may be inserted within a gene disrupting it while neighboring genes may show increased expression due to transactivation.

Neighboring genes that do not show upregulation may contain insulator-like elements in the upstream regions of the genes that interfere with transactivation. In an attempt to identify such elements, transcript levels of genes flanking T-DNA insertions in three activation-tagged lines, hat1, hat4, and hat7 were assessed.

In hat1, the T-DNA was inserted in At4g15290, a Cellulose synthase-like gene (CSL). The gene downstream of CSL, At4g15280, a UDP-glucosyl transferase (UGT), was strongly upregulated in the mutant hat1 compared to the wild-type plant. The gene upstream of CSL, At4g15300, a Cytochrome P450 (CYP), did not show any change in expression levels in hat1 compared to the wild-type plant. A 2-kb sequence upstream of the 1-kb promoter of CYP was selected as a region that contained the putative insulator-like element(s). The region was sub-divided into four sections of 500 bp each and named INS1, INS2, INS3 and INS4, respectively. Similarly, a 2-kb sequence upstream of the 1-kb promoter of UGT was identified as a region that would not contain any insulator-like elements and sub-divided into four 500 bp sequences named as INS5, INS6, INS7, and INS8, respectively.

Two independent mutant lines, hat4 and hat7, had the T-DNA insertion in the intergenic region between At1g60140, a Trehalose synthase-like gene (TSL) and At1g60160, a Potassium transporter family gene (PTF). Transcript analysis revealed upregulation of PTF, while TSL expression levels did not change in the mutants compared to the wild-type plants. A 2-kb sequence upstream of the 1-kb promoter of TSL was selected as a region that contained the putative insulator-like element(s). The region was sub-divided into four sections of 500 bp each and named INS9, INS10, INS 11 and INS12, respectively.

Each of the putative insulator-like sequences were cloned into the SpeI restriction site of a Gateway entry vector comprising of a CaMV35S enhancer upstream of a LTP2 promoter driving DS-RED, terminated with a CaMV 35S terminator. Cloning the putative insulator-like sequence in the SpeI site resulted in the CaMV35S enhancer and the LTP2 promoter now being separated from each other by the sequence. This entry vector was cloned into a destination vector using LR clonase, along with entry vectors carrying a ZM-PLTP::ZM-WUS2 cassette and a ZM-PLTP::ZM-ODP2 cassette to create an expression vector for transformation of maize immature embryos.

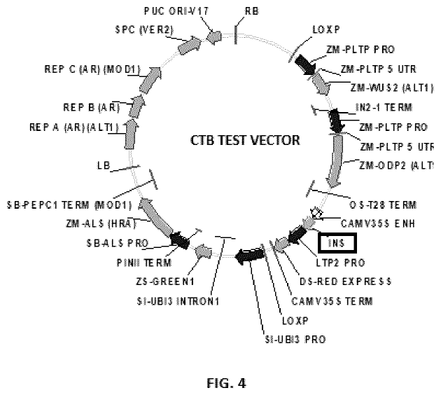

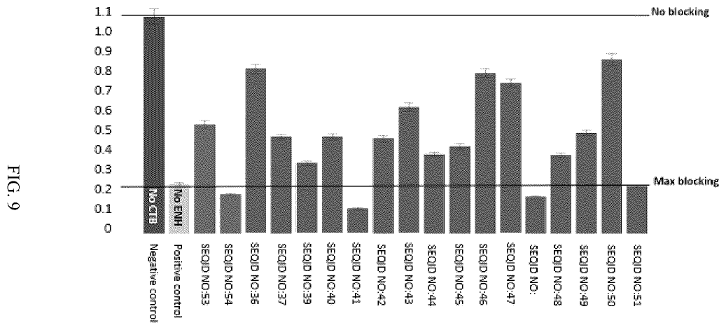

An example of a test vector is depicted in FIG. 4. Results from testing 19 unique CTB sequences are presented in FIG. 9.

### Example 5: Testing CTB-Like Candidates in Agrobacterium-Mediated Transformation of Maize Immature Embryos

Maize immature embryos were transformed with Agrobacterium harboring expression vectors (FIG. 4) carrying different CTB candidate sequences, in addition to control sequences of 500 bp length such as the Lotus japonicus Ubiquitin Terminator (INS16), or an expression vector without the CTB-like sequence (INS17). Two days after infection the immature embryos were transferred to resting medium for a week. Somatic embryos formed were observed under the fluorescence microscope for green and red fluorescence and photographed. Immature embryos, transformed with an expression vector with an insulator-like sequence, showing somatic embryos fluorescing green but not red were considered potential candidates with insulator-like activity, whereas constructs that fluoresced both green and red were considered negative for CTB-like activity.

Table 2 shows the results of testing CTB-like candidates in maize immature embryos. CTB activity resulted in absence of red fluorescence whereas no CTB activity resulted in the presence of red fluorescence. Green fluorescence being part of the CTB T-DNA used for transformation of maize immature embryos was present in all the tested samples.

TABLE 2

| | Green | Red | |
|---|---|---|---|
| CTB | Fluorescence | Flourescence | Result |
| INS1 | + | − | CTB activity |
| INS2 | + | − | CTB activity |
| INS3 | + | + | No CTB activity |
| INS4 | + | − | CTB activity |
| INS5 | + | + | No CTB activity |
| INS6 | + | − | CTB activity |
| INS8 | + | + | No CTB activity |
| INS9 | + | − | CTB activity |
| INS10 | + | + | No CTB activity |
| INS11 | + | + | No CTB activity |
| INS16 (Non- | + | + | No CTB activity |
| insulator DNA) | | | |
| INS17 (No | + | + | No CTB activity |
| Insulator) | | | |

As shown in Table 2, INS1, INS2, INS4, INS6, INS9 showed insulator-like activity as indicated by the absence of the DS-RED fluorescence. The non-insulator control sequence INS16 and the expression vector without the CTB-like sequence (INS17) did not show insulator-like activity.

### Example 6: Testing CTB Candidates in Agrobacterium-Mediated Transformation of Maize Leaf Explants

Maize leaf explants were transformed with Agrobacterium containing expression vectors with different CTB sequences. Two construct configurations were used.

Construct Configuration A: RB+LOXP+AT-5-IV-2 INS+ZM-HSP17.7 PRO::MO-CRE::PINII TERM+CTB+NOS PRO::ZM-WUS2::IN2 TERM+3×ENH-UBI1 PRO::ZM-ODP2::OS-T28 TERM+LOXP+SB-UBI PRO::ZSGREEN1::OS-UBI TERM+SB-ALS PRO::ZM-ALS::SB-UBI TERM+LB, where different test CTB sequences replaced “CTB”. The plasmids used and the transformation results obtained are summarized in Table 3.

TABLE 3

| | | # of | # of T0 | % T0 |
|---|---|---|---|---|
| Plasmid | CTB | Seedlings | plants | plants |
| PHP96034 | AT-5-IV-2 | 23 | 0 | 0 |
| (SEQ ID NO: 145) | INS (Control) | | | |
| PHP101513 | ZM-T1S1C1 | 23 | 1 | 4 |
| (SEQ ID NO: 146) | | | | |
| PHP101514 | ZM-T1S2C3 | 23 | 3 | 13 |
| (SEQ ID NO: 147) | | | | |
| PHP101652 | ZM-T1S2C8 | 23 | 5 | 22 |
| (SEQ ID NO: 148) | | | | |
| PHP101653 | ZM-T1S2C9 | 24 | 4 | 17 |
| (SEQ ID NO: 149) | | | | |
| PHP101654 | ZM-T2S2C9 | 24 | 3 | 13 |
| (SEQ ID NO: 150) | | | | |

When PHP96034 (SEQ TD NO: 145) was used for transformation, no TO plants were recovered. However, with the use of sequences ZM-T1S1C1, ZM-T15S2C3, ZM-T15S2C8, ZM-T1S2C9, and ZM-T252C9 as CTBs upstream of the NOS:WUS cassette, TO plants were recovered ranging from a frequency of 4-22%.

Construct Configuration B: RB+LOXP+NOS PRO::ZM-WUS2::N2 TERM+3×ENH-UBJ1 PRO::ZM-ODP2::OS-T28 TERM+CTB+ZM-HSP17.7 PRO::MO-CRE::PINII TERM+LOXP+SB-UBI PRO::ZSGREEN1::OS-UBI TERM+SB-ALS PRO::ZM-ALS::SB-UBI TERM+LB, where different test CTB sequences replaced “CTB”. The plasmids used and the transformation results obtained are summarized in Table 4. Data are collected from 3 replicated experiments and represented as Mean 00 TO plants±Standard Error.

TABLE 4

| Plasmid | CTB | % T0 plants |
|---|---|---|
| PHP97883 | No CTB | 193 ± 24 |
| (SEQ ID NO: 151) | | |
| PHP96037 | AT-5-IV-2 INS | 375 ± 80 |
| (SEQ ID NO: 152) | | |
| PHP101595 | AT-4G15300-I INS | 580 ± 0 |
| (SEQ ID NO: 153) | | |
| PHP101271 | AT-4G15300-II INS | 277 ± 59 |
| (SEQ ID NO: 154) | | |
| PHP101811 | AT-4G15300-IV INS | 540 ± 0 |
| (SEQ ID NO: 155) | | |
| PHP101272 | AT-4G15280-II INS | 320 ± 50 |
| (SEQ ID NO: 156) | | |
| PHP101596 | AT-1G60140-I INS | 255 ± 15 |
| (SEQ ID NO: 157) | | |
| PHP101512 | AT-4G15290-I INS | 430 ± 90 |
| (SEQ ID NO: 158) | | |
| PHP101597 | AT-4G15290-IV INS | 265 ± 5 |
| (SEQ ID NO: 159) | | |
| PHP101308 | ZM-T2S2C8 CTB | 288 ± 40 |
| (SEQ ID NO: 160) | | |
| PHP101206 | ZM-T2S2C2-4 CTB | 379 ± 79 |
| (SEQ ID NO: 161) | | |
| PHP101207 | ZM-T2S2C5 CTB | 422 ± 65 |
| (SEQ ID NO: 162) | | |
| PHP101205 | ZM-T1S2C9-2 CTB | 215 ± 54 |
| (SEQ ID NO: 163) | | |
| PHP 101094 | ZM-T1S6C6 CTB | 242 ± 34 |
| (SEQ ID NO: 164) | | |

In the absence of a CTB sequence between the 3×ENH-UBI:ODP2 cassette and the immediately downstream HSP17.7:CRE cassette of the construct PUP97883 (SEQ ID NO: 151) transformation frequency was 193%. With the inclusion of CTB sequences AT-5-IV-2 INS, AT-4G15300-I INS, AT-4G15300-II INS, AT-4G15300-IV INS, AT-4G15280-II INS, AT-1G60140-I INS, AT-4G15290-I INS, AT-4G15290-IV INS, ZM-T1S2C9-2 CTB, ZM-T1S6C6 CTB, ZM-T2S2C8 CTB, ZM-T2S2C2-4 CTB, or ZM-T2S2C5 CTB, transformation frequency increased ranging from 215-580%.

Two additional construct configurations are used.

Construct configuration C: RB+LOXP+NOS PRO::ZM-WUS2::IN2 TERM+CTB+3×ENH-UBI1 PRO::ZM-ODP2::OS-T28 TERM+CTB+ZM-HSP17.7 PRO::MO-CRE::PINII TERM+LOXP+SB-UBI PRO::ZSGREEN1::OS-UBI TERM+SB-ALS PRO::ZM-ALS::SB-UBI TERM+LB, where different test CTB sequences replace “CTB”. CTB sequences are expected to stabilize the expression of gene cassettes surrounding the CTB.

Construct configuration D: RB+LOXP+CTB+NOS PRO::ZM-WUS2::IN2 TERM+3×ENH-UBI1 PRO::ZM-ODP2::OS-T28 TERM+CTB+ZM-HSP17.7 PRO::MO-CRE::PINII TERM+LOXP+SB-UBI PRO::ZSGREEN1::OS-UBI TERM+SB-ALS PRO::ZM-ALS::SB-UBI TERM+LB, where different test CTB sequences replace “CTB”. CTB sequences are expected to stabilize the expression of gene cassettes surrounding the CTB.

### Example 7: Effect of CTB's on Expression in a Gene Stack Configuration

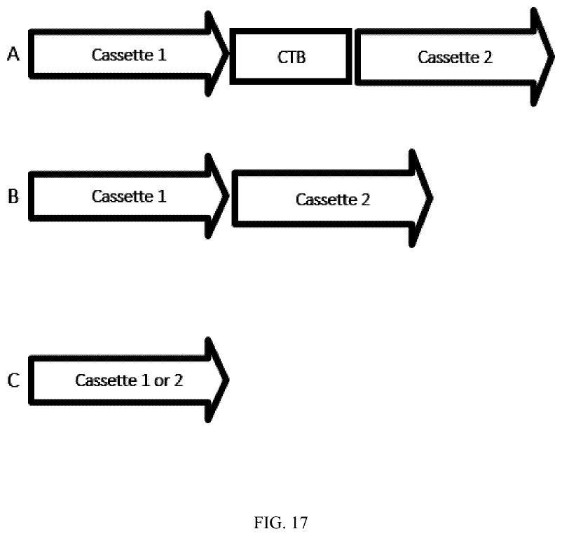

CTB sequences were tested for properties that prevent the down-regulation of one or both genes in a gene stack vector configuration consisting of two tandemly oriented expression cassettes (FIG. 17). Expression of the upstream cassette in the vector creates a situation that can result in a negative effect on the expression of the downstream cassette. Negative effects on the expression of the upstream cassette can also occur in these vectors. These impacts are apparent when expression is compared to control constructs where each cassette is expressed in separate vectors.

To determine if a CTB sequence had a positive effect on the expression of one or both cassettes in a stacked vector configuration, each CTB sequence was cloned between the expression cassettes and expressed in maize in a first pass analysis. Results are shown in Table 5.

TABLE 5

| CTB | First cassette | Second cassette |
|---|---|---|
| No CTB | 4 | 1 |
| ZM-T1S1C1 | 8 | 6 |
| (SEQ ID NO: 1) | | |
| ZM-T1S1C5 | 0 | 1 |
| (SEQ ID NO: 30) | | |
| ZM-T1S2C1 | 6 | 4 |
| (SEQ ID NO: 9) | | |
| ZM-T1S2C3 | 6 | 6 |
| (SEQ ID NO: 2) | | |
| ZM-T1S2C3-2 | 2 | 2 |
| (SEQ ID NO: 3) | | |
| ZM-T1S2C6 | 3 | 2 |
| (SEQ ID NO: 4) | | |
| ZM-T1S2C7 | 6 | 4 |
| (SEQ ID NO: 5) | | |
| ZM-T1S2C8 | 6 | 6 |
| (SEQ ID NO: 6) | | |
| ZM-T1S2C9 | 6 | 9 |
| (SEQ ID NO: 7) | | |
| ZM-T1S2C9-2 | 4 | 3 |
| (SEQ ID NO: 8) | | |
| ZM-T1S6C1 | 8 | 4 |
| (SEQ ID NO: 11) | | |
| ZM-T1S6C5 | 3 | 3 |
| (SEQ ID NO: 31) | | |
| ZM-T1S6C5-2 | 5 | 3 |
| (SEQ ID NO: 32) | | |
| ZM-T1S6C6 | 3 | 3 |
| (SEQ ID NO: 10) | | |
| ZM-T2S1C1 | 4 | 2 |
| (SEQ ID NO: 12) | | |
| ZM-T2S1C3 | 4 | 4 |
| (SEQ ID NO: 13) | | |
| ZM-T2S1C4 | 5 | 3 |
| (SEQ ID NO: 14) | | |
| ZM-T2S1C5 | 4 | 3 |
| (SEQ ID NO: 15) | | |
| ZM-T2S1C5-2 | 2 | 1 |
| (SEQ ID NO: 32) | | |
| ZM-T2S1C5-3 | 3 | 1 |
| (SEQ ID NO: 33) | | |
| ZM-T2S2C2 | 6 | 6 |
| (SEQ ID NO: 20) | | |
| ZM-T2S2C2-2 | 4 | 1 |
| (SEQ ID NO: 22) | | |
| ZM-T2S2C2-3 | 1 | 2 |
| (SEQ ID NO: 23) | | |
| ZM-T2S2C2-4 | 2 | 3 |
| (SEQ ID NO: 24) | | |
| ZM-T2S2C3 | 1 | 3 |
| (SEQ ID NO: 21) | | |
| ZM-T2S2C4 | 5 | 4 |
| (SEQ ID NO: 18) | | |
| ZM-T2S2C4-2 | 2 | 3 |
| (SEQ ID NO: 19) | | |
| ZM-T2S2C5 | 2 | 2 |
| (SEQ ID NO: 26) | | |
| ZM-T2S2C5-2 | 1 | 0 |
| (SEQ ID NO: 27) | | |
| ZM-T2S2C5-3 | 4 | 7 |
| (SEQ ID NO: 28) | | |
| ZM-T2S2C5-4 | 3 | 0 |
| (SEQ ID NO: 29) | | |
| ZM-T2S2C5-5 | 9 | 9 |
| (SEQ ID NO: 35) | | |
| ZM-T2S2C7 | 5 | 4 |
| (SEQ ID NO: 25) | | |
| ZM-T2S2C8 | 5 | 4 |
| (SEQ ID NO: 16) | | |
| ZM-T2S2C9 | 5 | 6 |
| (SEQ ID NO: 17) | | |

0-9 scale based on the raw data first being normalized to the respective single gene control, then ranked based on the highest and lowest values.
0 = expression is 40%-50% of the single gene control
9 = expression is >130% of the single gene control

A subset of the CTB's were advanced for additional analysis in stably transformed corn plants. Results in V6 leaf tissue are shown in Table 6 and for R1 stalk in Table 7.

TABLE 6

| CTB | First Cassette (leaf) | Second Cassette (leaf) |
|---|---|---|
| empty | 5 | 9 |
| ZM-T1S1C1 | 5 | 5 |
| (SEQ ID NO: 1) | | |
| ZM-T1S2C3 | 2 | 3 |
| (SEQ ID NO: 2) | | |
| ZM-T1S2C3-2 | 3 | 3 |
| (SEQ ID NO: 3) | | |
| ZM-T1S2C8 | 2 | 3 |
| (SEQ ID NO: 6) | | |
| ZM-T1S2C9 | 4 | 4 |
| (SEQ ID NO: 7) | | |
| ZM-T2S1C5 | 1 | 1 |
| (SEQ ID NO: 15) | | |
| ZM-T2S2C9 | 1 | 2 |
| (SEQ ID NO: 17) | | |

0-9 scale based on the raw data first being normalized to the respective single gene control, then ranked based on the highest and lowest values.
0 = expression is 40%-50% of the single gene control
1 = expression is 50%-60% of the single gene control
2 = expression is 60%-70% of the single gene control
3 = expression is 70%-80% of the single gene control
4 = expression is 80%-90% of the single gene control
5 = expression is 90%-100% of the single gene control
6 = expression is 100%-110% of the single gene control
7 = expression is 110%-120% of the single gene control
8 = expression is 120%-130% of the single gene control
9 = expression is >130% of the single gene control

TABLE 7

| CTB | First Cassette (stalk) | Second Cassette (stalk) |
|---|---|---|
| empty | 2 | 5 |
| ZM-T1S1C1 | 4 | 5 |
| (SEQ ID NO: 1) | | |
| ZM-T1S2C3 | 4 | 5 |
| (SEQ ID NO: 2) | | |
| ZM-T1S2C3-2 | 3 | 4 |
| (SEQ ID NO: 3) | | |
| ZM-T1S2C8 | 4 | 4 |
| (SEQ ID NO: 6) | | |
| ZM-T1S2C9 | 4 | 6 |
| (SEQ ID NO: 7) | | |
| ZM-T2S1C5 | 4 | 6 |
| (SEQ ID NO: 15) | | |
| ZM-T2S2C9 | 5 | 5 |
| (SEQ ID NO: 17) | | |

0-9 scale based on the raw data first being normalized to the respective single gene control, then ranked based on the highest and lowest values.
0 = expression is 40%-50% of the single gene control
1 = expression is 50%-60% of the single gene control
2 = expression is 60%-70% of the single gene control
3 = expression is 70%-80% of the single gene control
4 = expression is 80%-90% of the single gene control
5 = expression is 90%-100% of the single gene control
6 = expression is 100%-110% of the single gene control
7 = expression is 110%-120% of the single gene control
8 = expression is 120%-130% of the single gene control
9 = expression is >130% of the single gene control

Sixteen CTBs identified from the Arabidopsis activation-tagged lines were tested for their impact on the expression of the upstream and downstream cassettes in a gene stack configuration by placing the CTB between tandemly oriented expression cassettes (FIG. 17). Results from expression in maize in a first pass analysis are shown in Table 8.

TABLE 8

| CTB | First Cassette | Second Cassette |
|---|---|---|
| No CTB | 3 | 2 |
| INS1 | 1 | 1 |
| (SEQ ID NO: 36) | | |
| INS2 | 3 | 4 |
| (SEQ ID NO: 37) | | |
| INS3 | 0 | 0 |
| (SEQ ID NO: 38) | | |
| INS4 | 9 | 8 |
| (SEQ ID NO: 39) | | |
| INS5 | 0 | 0 |
| (SEQ ID NO: 40) | | |
| INS6 | 3 | 4 |
| (SEQ ID NO: 41) | | |
| INS7 | 1 | 3 |
| (SEQ ID NO: 42) | | |
| INS8 | 1 | 1 |
| (SEQ ID NO: 43) | | |
| INS9 | 1 | 2 |
| (SEQ ID NO: 44) | | |
| INS10 | 2 | 2 |
| (SEQ ID NO: 45) | | |
| INS11 | 2 | 4 |
| (SEQ ID NO: 46) | | |
| INS12 | 2 | 4 |
| (SEQ ID NO: 47) | | |
| INS24 | 2 | 2 |
| (SEQ ID NO: 48) | | |
| INS25 | 0 | 0 |
| (SEQ ID NO: 49) | | |
| INS26 | 0 | 0 |
| (SEQ ID NO: 50) | | |
| INS27 | 4 | 5 |
| (SEQ ID NO: 51) | | |

0-9 scale based on the raw data first being normalized to the respective single gene control, then ranked based on the highest and lowest values.
0 = expression is 40%-50% of the single gene control
1 = expression is 50%-60% of the single gene control
2 = expression is 60%-70% of the single gene control
3 = expression is 70%-80% of the single gene control
4 = expression is 80%-90% of the single gene control
5 = expression is 90%-100% of the single gene control
6 = expression is 100%-110% of the single gene control
7 = expression is 110%-120% of the single gene control
8 = expression is 120%-130% of the single gene control
9 = expression is >130% of the single gene control

Similar results were obtained from experiments in other tissue types including R1 silk, leaf, and husk.

As evident from the results above, where gene expression in a stack configuration is affected by adjacent cassettes, several CTB candidates were able to reduce the negative effects on gene expression.

### Example 8: Testing CTB-Like Candidates in Peg-Mediated Transformation of Maize Leaf Protoplasts

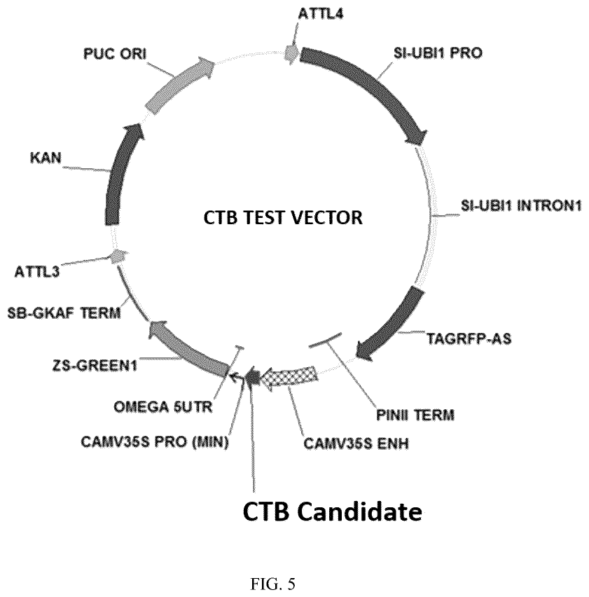

Protoplasts were isolated from leaf mesophyll cells from 7-day old etiolated maize seedlings using a modified protocol disclosed in (Sheen, Plant Physiol. 127: 1466-1475, 2001). Around 5 pmol of DNA (FIG. 5) was transfected into the protoplasts using 40% PEG. Transfected protoplasts were incubated at room temperature for 16 hours. The constitutive red fluorescence (TAG-RFP) was used for normalization while the CaMV35S enhancer and minimal promoter along with the putative insulator-like sequence were used to drive green fluorescence (ZS-GREEN). Fluorescence of both proteins was quantified using an automated inverted microscope (Biotek Cytation 5). Fluorescence was measured at the individual protoplast level, the green fluorescence was normalized to the red fluorescence, and geometric mean was calculated for all protoplasts (˜2000-3000) in the transfection.

Together, the CaMV35S enhancer and minimal promoter drove strong expression of ZS-GREEN in the protoplasts. In the absence of the enhancer, the minimal 35S promoter produced expression levels that were not detectable in the current system. Results from testing 18 unique CTB sequences identified from Arabidopsis and one synthetic sequence (AT-5-IV-8 CTB) using maize leaf protoplasts are presented in Table 9. Table 9 shows the expression of a reporter gene in maize leaf protoplasts in the presence or absence of CTBs. Results are presented as the average (AVG) of the geometric mean from two replicates and the Standard Deviation (STD).

TABLE 9

| | | | | Geo | Geo | Geo | Geo |
|---|---|---|---|---|---|---|---|
| | | | Reporter | Mean | Mean | Mean | Mean |
| Class | Enhancer | CTB | Promoter | Rep1 | Rep2 | AVG | STD |
| Control - | CAMV35S | None | CAMV35S | 1.071 | 0.996 | 1.033 | 0.053 |
| No CTB | ENH | | PRO (MIN) | | | | |
| Control - | None | None | CAMV35S | 0.254 | 0.219 | 0.237 | 0.024 |
| No ENH | | | PRO (MIN) | | | | |
| Arabidopsis | CAMV35S | AT-5-IV-2 | CAMV35S | 0.556 | 0.484 | 0.520 | 0.051 |
| | ENH | CTB | PRO (MIN) | | | | |
| Arabidopsis | CAMV35S | AT-5-IV-7 | CAMV35S | 0.199 | 0.178 | 0.188 | 0.015 |
| | ENH | CTB | PRO (MIN) | | | | |
| Arabidopsis | CAMV35S | AT-4G15300- | CAMV35S | 0.882 | 0.692 | 0.787 | 0.134 |
| | ENH | I CTB | PRO (MIN) | | | | |
| Arabidopsis | CAMV35S | AT-4G15300- | CAMV35S | 0.482 | 0.441 | 0.462 | 0.029 |
| | ENH | II CTB | PRO (MIN) | | | | |
| Arabidopsis | CAMV35S | AT-4G15300- | CAMV35S | 0.352 | 0.322 | 0.337 | 0.021 |
| | ENH | IV CTB | PRO (MIN) | | | | |
| Arabidopsis | CAMV35S | AT-4G15280- | CAMV35S | 0.516 | 0.410 | 0.463 | 0.075 |
| | ENH | I CTB | PRO (MIN) | | | | |
| Arabidopsis | CAMV35S | AT-4G15280- | CAMV35S | 0.126 | 0.115 | 0.121 | 0.008 |
| | ENH | II CTB | PRO (MIN) | | | | |
| Arabidopsis | CAMV35S | AT-4G15280- | CAMV35S | 0.453 | 0.454 | 0.454 | 0.000 |
| | ENH | III CTB | PRO (MIN) | | | | |
| Arabidopsis | CAMV35S | AT-4G15280- | CAMV35S | 0.627 | 0.580 | 0.603 | 0.034 |
| | ENH | IV CTB | PRO (MIN) | | | | |
| Arabidopsis | CAMV35S | AT-1G60140- | CAMV35S | 0.422 | 0.333 | 0.377 | 0.063 |
| | ENH | I CTB | PRO (MIN) | | | | |
| Arabidopsis | CAMV35S | AT-1G60140- | CAMV35S | 0.441 | 0.392 | 0.416 | 0.035 |
| | ENH | II CTB | PRO (MIN) | | | | |
| Arabidopsis | CAMV35S | AT-1G60140- | CAMV35S | 0.713 | 0.818 | 0.766 | 0.074 |
| | ENH | III CTB | PRO (MIN) | | | | |
| Arabidopsis | CAMV35S | AT-1G60140- | CAMV35S | 0.692 | 0.745 | 0.718 | 0.038 |
| | ENH | IV CTB | PRO (MIN) | | | | |
| Arabidopsis | CAMV35S | AT-4G15290- | CAMV35S | 0.368 | 0.381 | 0.375 | 0.009 |
| | ENH | I INS | PRO (MIN) | | | | |
| Arabidopsis | CAMV35S | AT-4G15290- | CAMV35S | 0.473 | 0.490 | 0.482 | 0.012 |
| | ENH | II INS | PRO (MIN) | | | | |
| Arabidopsis | CAMV35S | AT-4G15290- | CAMV35S | 0.732 | 0.929 | 0.830 | 0.139 |
| | ENH | III INS | PRO (MIN) | | | | |
| Arabidopsis | CAMV35S | AT-4G15290- | CAMV35S | 0.225 | 0.226 | 0.226 | 0.001 |
| | ENH | IV INS | PRO (MIN) | | | | |
| Arabidopsis | CAMV35S | AT-5-III-1 | CAMV35S | 0.207 | 0.147 | 0.177 | 0.042 |
| | ENH | | PRO (MIN) | | | | |
| Synthetic | CAMV35S | AT-5-IV-8 | CAMV35S | 0.206 | 0.177 | 0.192 | 0.021 |
| | ENH | CTB | PRO (MIN) | | | | |

Results from testing 35 unique CTB sequences identified from maize genome mining and 5 combinations of 2 sequences in tandem, using maize leaf protoplasts are presented in Table 10. Table 10 shows the expression of a reporter gene in maize leaf protoplasts in the presence or absence of CTBs. Results are presented as the average (AVG) of the geometric mean from two replicates and the Standard Deviation (STD).

TABLE 10

| | | | | Geo | Geo | Geo | Geo |
|---|---|---|---|---|---|---|---|
| | | | Reporter | Mean | Mean | Mean | Mean |
| Class | Enhancer | CTB | Promoter | Rep1 | Rep2 | AVG | STD |
| Control - | CAMV35S | None | CAMV35S | 1.217 | 1.291 | 1.254 | 0.053 |
| No CTB | ENH | | PRO (MIN) | | | | |
| Control - | None | None | CAMV35S | 0.236 | 0.224 | 0.230 | 0.008 |
| No ENH | | | PRO (MIN) | | | | |
| Zea mays | CAMV35S | ZM-T1S1C1 | CAMV35S | 0.440 | 0.390 | 0.415 | 0.035 |
| | ENH | CTB | PRO (MIN) | | | | |
| Zea mays | CAMV35S | ZM-T1S2C3 | CAMV35S | 0.501 | 0.466 | 0.483 | 0.024 |
| | ENH | CTB | PRO (MIN) | | | | |
| Zea mays | CAMV35S | ZM-T1S2C3- | CAMV35S | 0.419 | 0.370 | 0.395 | 0.035 |
| | ENH | 2 CTB | PRO (MIN) | | | | |
| Zea mays | CAMV35S | ZM-T1S2C6 | CAMV35S | 0.441 | 0.412 | 0.427 | 0.020 |
| | ENH | CTB | PRO (MIN) | | | | |
| Zea mays | CAMV35S | ZM-T1S2C7 | CAMV35S | 0.374 | 0.280 | 0.327 | 0.066 |
| | ENH | CTB | PRO (MIN) | | | | |
| Zea mays | CAMV35S | ZM-T1S2C8 | CAMV35S | 0.443 | 0.424 | 0.433 | 0.014 |
| | ENH | CTB | PRO (MIN) | | | | |
| Zea mays | CAMV35S | ZM-T1S2C9 | CAMV35S | 0.925 | 0.735 | 0.830 | 0.134 |
| | ENH | CTB | PRO (MIN) | | | | |
| Zea mays | CAMV35S | ZM-T1S2C9- | CAMV35S | 0.126 | 0.113 | 0.120 | 0.009 |
| | ENH | 2 CTB | PRO (MIN) | | | | |
| Zea mays | CAMV35S | ZM-T1S2C1 | CAMV35S | 0.752 | 0.626 | 0.689 | 0.088 |
| | ENH | CTB | PRO (MIN) | | | | |
| Zea mays | CAMV35S | ZM-T1S6C6 | CAMV35S | 0.342 | 0.277 | 0.310 | 0.046 |
| | ENH | CTB | PRO (MIN) | | | | |
| Zea mays | CAMV35S | ZM-T1S6C1 | CAMV35S | 0.947 | 0.817 | 0.882 | 0.092 |
| | ENH | CTB | PRO (MIN) | | | | |
| Zea mays | CAMV35S | ZM-T2S1C1 | CAMV35S | 0.736 | 0.535 | 0.636 | 0.142 |
| | ENH | CTB | PRO (MIN) | | | | |
| Zea mays | CAMV35S | ZM-T2S1C3 | CAMV35S | 0.626 | 0.540 | 0.583 | 0.061 |
| | ENH | CTB | PRO (MIN) | | | | |
| Zea mays | CAMV35S | ZM-T2S1C4 | CAMV35S | 0.841 | 0.830 | 0.836 | 0.008 |
| | ENH | CTB | PRO (MIN) | | | | |
| Zea mays | CAMV35S | ZM-T2S1C5 | CAMV35S | 1.194 | 0.925 | 1.060 | 0.190 |
| | ENH | CTB | PRO (MIN) | | | | |
| Zea mays | CAMV35S | ZM-T2S2C8 | CAMV35S | 0.263 | 0.192 | 0.227 | 0.050 |
| | ENH | CTB | PRO (MIN) | | | | |
| Zea mays | CAMV35S | ZM-T2S2C9 | CAMV35S | 1.345 | 1.115 | 1.230 | 0.163 |
| | ENH | CTB | PRO (MIN) | | | | |
| Zea mays | CAMV35S | ZM-T2S2C4 | CAMV35S | 2.147 | 1.887 | 2.017 | 0.183 |
| | ENH | CTB | PRO (MIN) | | | | |
| Zea mays | CAMV35S | ZM-T2S2C4- | CAMV35S | 0.864 | 0.740 | 0.802 | 0.088 |
| | ENH | 2 CTB | PRO (MIN) | | | | |
| Zea mays | CAMV35S | ZM-T2S2C2 | CAMV35S | 1.281 | 1.252 | 1.266 | 0.021 |
| | ENH | CTB | PRO (MIN) | | | | |
| Zea mays | CAMV35S | ZM-T2S2C3 | CAMV35S | 0.601 | 0.564 | 0.583 | 0.026 |
| | ENH | CTB | PRO (MIN) | | | | |
| Zea mays | CAMV35S | ZM-T2S2C2- | CAMV35S | 0.859 | 0.979 | 0.919 | 0.084 |
| | ENH | 2 CTB | PRO (MIN) | | | | |
| Zea mays | CAMV35S | ZM-T2S2C2- | CAMV35S | 0.944 | 0.819 | 0.882 | 0.088 |
| | ENH | 3 CTB | PRO (MIN) | | | | |
| Zea mays | CAMV35S | ZM-T2S2C2- | CAMV35S | 0.182 | 0.150 | 0.166 | 0.023 |
| | ENH | 4 CTB | PRO (MIN) | | | | |
| Zea mays | CAMV35S | ZM-T2S2C7 | CAMV35S | 0.553 | 0.489 | 0.521 | 0.045 |
| | ENH | CTB | PRO (MIN) | | | | |
| Zea mays | CAMV35S | ZM-T2S2C5 | CAMV35S | 0.356 | 0.351 | 0.354 | 0.004 |
| | ENH | CTB | PRO (MIN) | | | | |
| Zea mays | CAMV35S | ZM-T2S2C5- | CAMV35S | 0.898 | 1.060 | 0.979 | 0.115 |
| | ENH | 2 CTB | PRO (MIN) | | | | |
| Zea mays | CAMV35S | ZM-T2S2C5- | CAMV35S | 0.396 | 0.390 | 0.393 | 0.004 |
| | ENH | 3 CTB | PRO (MIN) | | | | |
| Zea mays | CAMV35S | ZM-T2S2C5- | CAMV35S | 0.972 | 0.875 | 0.924 | 0.069 |
| | ENH | 4 CTB | PRO (MIN) | | | | |
| Zea mays | CAMV35S | ZM-T1S1C5 | CAMV35S | 1.5715 | 0.9535 | 1.262 | 0.437 |
| | ENH | CTB | PRO (MIN) | | | | |
| Zea mays | CAMV35S | ZM-T1S6C5 | CAMV35S | 0.7203 | 0.5131 | 0.617 | 0.147 |
| | ENH | CTB | PRO (MIN) | | | | |
| Zea mays | CAMV35S | ZM-T2S1C5- | CAMV35S | 0.7849 | 0.6100 | 0.697 | 0.124 |
| | ENH | 2 CTB | PRO (MIN) | | | | |
| Zea mays | CAMV35S | ZM-T2S1C5- | CAMV35S | 0.7853 | 0.4706 | 0.628 | 0.222 |
| | ENH | 3 CTB | PRO (MIN) | | | | |
| Zea mays | CAMV35S | ZM-T1S6C5- | CAMV35S | 3.2063 | 2.6999 | 2.953 | 0.358 |
| | ENH | 2 CTB | PRO (MIN) | | | | |
| Zea mays | CAMV35S | ZM-T2S2C5- | CAMV35S | 1.4667 | 1.2385 | 1.353 | 0.161 |
| | ENH | 5 CTB | PRO (MIN) | | | | |
| Zea mays | CAMV35S | ZM-T1S1C5 | CAMV35S | 1.5929 | 2.0715 | 1.832 | 0.338 |
| | ENH | CTB//ZM- | PRO (MIN) | | | | |
| | | T2S2C5-5 | | | | | |
| | | CTB | | | | | |
| Zea mays | CAMV35S | ZM-T1S6C5 | CAMV35S | 1.0629 | 0.7369 | 0.900 | 0.231 |
| | ENH | CTB//ZM- | PRO (MIN) | | | | |
| | | T1S6C5-2 | | | | | |
| | | CTB | | | | | |
| Zea mays | CAMV35S | ZM-T2S1C5- | CAMV35S | 1.0021 | 0.7841 | 0.893 | 0.154 |
| | ENH | 2 CTB//ZM- | PRO (MIN) | | | | |
| | | T2S1C5-3 | | | | | |
| | | CTB | | | | | |
| Zea mays | CAMV35S | ZM-T1S2C3 | CAMV35S | 0.6999 | 0.4759 | 0.588 | 0.158 |
| | ENH | TRB//ZM- | PRO (MIN) | | | | |
| | | T2S2C3 CTB | | | | | |
| Zea mays | CAMV35S | ZM-T2S2C4 | CAMV35S | 2.8680 | 2.3108 | 2.589 | 0.394 |
| | ENH | TRB//ZM- | PRO (MIN) | | | | |
| | | T2S2C4-2 | | | | | |
| | | CTB | | | | | |

### Example 9: Endogenous DNA CTB Elements for Stable Transgenes Performance in Breeding Products

The performance of transgenes can vary significantly in different germplasm or environments due to the interactions of transgene×genetics or transgene×genetics×environments. Thus, a breeding program must conduct thorough trait evaluations in different germplasm and environments. One hypothesis of trait variation across germplasm and environments is due to specific regulatory elements existing in specific genetics and causing unfavorable interactions. For example, the nearby or distal endogenous enhancers could unfavorably increase the level of transgene expression and cause unintended agronomic consequences. In addition, plant genomes often contain large fraction of transposon elements which can cause unintended transgene silencing.

This example is about a novel trait design concept and application of CTB identification and elements to improve the robustness of transgene performance across different germplasm and environments by preventing or mitigating the transgene×genetics interaction or transgene×genetics×environments interaction. CTB is one type of regulatory element in genome to preserve the gene expression level of their target genes by two possible modes of actions or both. One mode of action is called enhancer-blocking effect and the other is silence barrier effect.

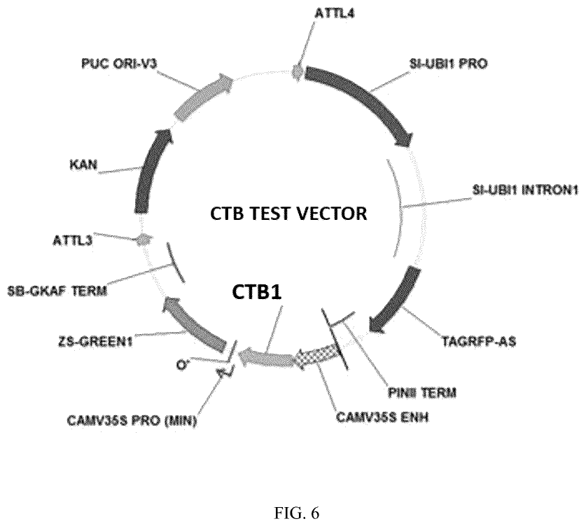

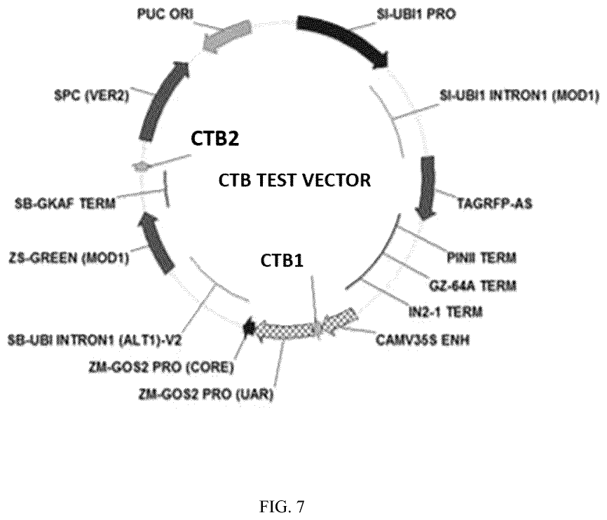

The identified endogenous CTB elements in crop genomes can be placed as a single insulator element (FIG. 6) or in pair for the traits of interest (FIG. 7). A custom computational workflow was developed to identify the maize endogenous insulator elements based on the gene expression and chromatin loop data. The experimental chromatin loop data can detect the DNA interaction between the target genes and their regulatory elements. More than 800 putative insulator elements were identified by computational search and 40 CTBs or CTB pairs are being tested in the protoplast system for validation.

The validated insulator will enable the trait performance independent on the genetics and environments so that the transgenes are robust to broad germplasm and environments. The successful deployment of insulator element in trait product means significant operation cost saving with stable trait performance.

### Example 10: CTB Vectors for Soy Transformation

Four CTB vectors (B-E) were built for soybean transformation. Each vector contained four identical expression cassettes. The first cassette comprised a Cre recombinase gene under the control of soybean heat-shock GmHSP17.3B promoter (“CRE Cassette”) for excision. The second cassette comprised a spectinomycin-resistance SPCN gene as a plant selectable marker (“SPCN Cassette”). The third cassette comprised DsRED as a visual marker in transformed plant cells (“DsRed Cassette”). The fourth cassette comprised an insecticidal protein gene as an exemplary trait gene (“Trait Cassette”). The insulator candidates in vectors B-D flanked the Cre Cassette. Vector A, used as a negative control, comprised the four identical expression cassettes but lacked insulator candidates (no insulation). The insulator configurations and candidates tested, namely, AT-5-IV-2 INS, AT-5-III-1 INS, AT-5-IV-7 INS, are shown in Table 11.

TABLE 11

| Vec- | |
|---|---|
| tor | Description |
| A | RB + LOXP + Cre Cassette + SPCN Cassette + DsRed Cassette + |
| | LOXP + Trait Cassette + LB |
| B | RB + LOXP + AT-5-IV-2 INS + Cre Cassette + AT-5-IV-2 INS + |
| | SPCN Cassette + DsRed Cassette + LOXP + Trait Cassette + LB |
| C | RB + LOXP + AT-5-III-1 INS + Cre Cassette + AT-5-IV-7 INS + |
| | SPCN Cassette + DsRed Cassette + LOXP + Trait Cassette + LB |
| D | RB + LOXP + AT-5-IV-2 INS + Cre Cassette + AT-5-III-1 INS + |
| | SPCN Cassette + DsRed Cassette + LOXP + Trait Cassette + LB |
| E | RB + LOXP + AT-5-IV-7 INS + Cre Cassette + AT-5-III-1 INS + |
| | SPCN Cassette + DeRed Cassette + LOXP + Trait Cassette + LB |

Mature dry seed from soybean 93Y21 cultivar was surface-sterilized for 16 hours using chlorine gas, produced by mixing 3.5 mL of 12 N HCl with 100 mL of commercial bleach (5.25% sodium hypochloride), as described by Di et al. ((1996) Plant Cell Rep 15:746-750). Disinfected seeds were imbibed on semi-solid medium containing 5 g/l sucrose and 6 g/l agar at room temperature in the dark. After 6-8 hours imbibition, the seeds were soaked in sterile distilled water at room temperature in the dark for overnight (˜16 hrs). Intact embryonic axes (EA) were isolated from the imbibed seeds. Ochrobactrum-mediated EA transformation was carried out as described below.

Ochrobactrum haywardense H1 lines containing the vectors listed in Table 11 were used for transformation. A volume of 15 mL of Ochrobactrum haywardense H1 suspension (OD 0.5 at 600 nm) in infection medium composed of 1/10× Gamborg B5 basal medium, 30 g/L sucrose, 20 mM MES, 0.25 mg/L GA3, 1.67 mg/L BAP, 200 μM Acetosyringone and 1 mM DTT in PH 5.4) was added to about 200-300 EAs in 25×100 mm petri plates. The plates were sealed with parafilm (“Parafilm M” VWR Cat #52858), then sonicated (Sonicator-VWR model 50T) for 30 seconds. After sonication, the EAs were incubated 2 hrs at room temperature. After incubation, the excess bacterial suspension was removed and about 200-300 EAs were transferred to a single layer of autoclaved sterile filter paper (VWR #415/Catalog #28320-020) in 25×100 mm petri plates. The plates were sealed with Micropore tape (Catalog #1530-0, 3M, St. Paul, MN, USA) and incubated under dim light (1-2 E/m²/s), cool white fluorescent lamps for 16 hours/day at 21° C. for 3 days. After co-cultivation, the base of each EA was embedded in shoot induction medium (Production #R7100, PhytoTech Labs, Shawnee, KS, USA) containing 30 g/L sucrose, 6 g/L agar and 25 mg/L Spectinomycin (PhytoTech Labs) as a selectable agent and 500 mg/L cefotaxime (GoldBio, ST Louis, MO, USA). Shoot induction was carried out in a Percival Biological Incubator or growth room at 26° C. with a photoperiod of 16 hours and a light intensity of 60-100 E/m²/s.

After 5-6 weeks in selection medium, the spectinomycin-resistant shoots were counted to calculate transformation frequencies (Table 12). Transformation frequencies of vectors B-D ranged from 19.8%-31.3%, while the transformation frequency of control vector A (no insulation) was 30%.

TABLE 12

| Vector | Total #EA Transformed | # SPCN Resistant Shoots Recovered | |
|---|---|---|---|
| A | 1530 | 459 | (30%) |
| B | 2310 | 725 | (31.3%) |
| C | 1910 | 380 | (19.8%) |
| D | 1920 | 492 | (25.6%) |
| E | 1830 | 305 | (16.6%) |

Alternative experiments are contemplated.

In one experiment, CTB candidates are cloned between the CAMV35S enhancer and 35S minimal promoter or between the CAMV35S enhancer and 35S minimal promoter, and UBQ3 terminator in a TagRFP expression cassette. The negative control (no insulation) is a vector with no insulator and the positive control (max insulation) is a vector with only the 35S promoter (e.g. no CAMV35S enhancer). The CAMV35S minimal promoter produces no TagRFP fluorescence in the absence an enhancer.

In another experiment, these vectors are tested in various dicot plants such as Ochrobactrum-mediated soybean transformation, Agrobacterium rhizogenes-mediated soybean hairy root transformation system (Cho et al. High-efficiency induction of soybean hairy roots and propagation of the soybean cyst nematode, Planta, 210, 195-204. 2000), or Agrobacterium tumefaciens-mediated alfalfa, canola, cotton, soybean, and sunflower transformation. The quantification of fluorescence is performed using Zeica fluorescent microscope in transiently and stably transformed shoots and hairy roots in dicot plants to evaluate CTB candidate performance.

### Example 11: Motifs Enriched Among Selected CTBs

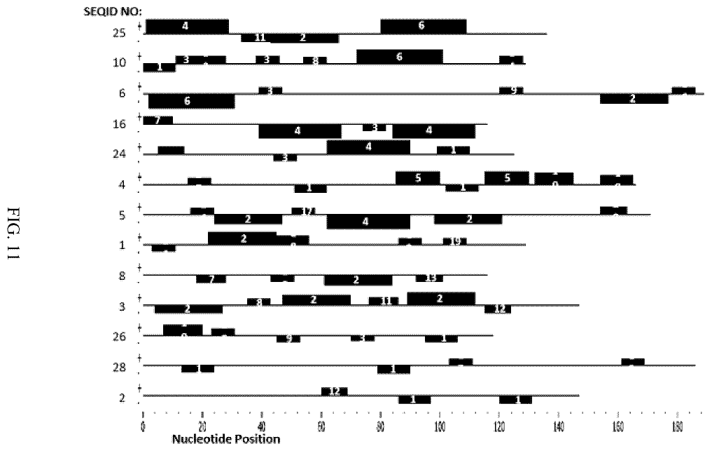

Thirteen CTB candidates were selected for motif analysis using a Motif Alignment and Search Tool (MAST version 5.1.1; Timothy L. Bailey and Michael Gribskov, “Combining evidence using p-values: application to sequence homology searches”, Bioinformatics, 14(1):48-54, 1998). Motifs that were identified are described in Table 13. Representative sequences comprising these motifs are shown in FIG. 11. U=T # alias Uracil to Thymine (permit U in input sequences); R=AG; Y=CT; K=GT; M=AC; S=CG; W=AT; B=CGT; D=GAT; H=ACT; V=ACG; N=ACGT # wildcard symbol.

TABLE 13

| Motif | | SEQ |
|---|---|---|
| ID: | Motif Sequence | ID NO: |
| 1 | TGTTTTTYTCT | 133 |
| 2 | GDTGARGADGGACNVSGYGSHNG | 134 |
| 3 | CGGCCCAA | |
| 4 | RKGCCATANAGYSBDRCCMKTCSAYCGT | 135 |
| 5 | ATTCACGAGGTAGCC | 136 |
| 6 | GVYCRSMKSKTTSVTGSGAYYCBGCVRAC | 137 |
| 7 | GCSSGGGGCA | 138 |
| 8 | CCAACCGA | |
| 9 | TTCCAGGC | |
| 10 | AGAGATGTKTGGA | 139 |
| 11 | GGGASCWCGA | 140 |
| 12 | CGTGCGRAA | |
| 13 | GCCCAAAMC | |
| 14 | CCCCYACC | |
| 15 | GGTCCATT | |
| 16 | SACGCSGC | |
| 17 | AACCATCA | |
| 18 | GGSWGWGGAGG | 141 |
| 19 | TCCTTGSC | |
| 20 | GSTGAAACG | |

### Example 12: Production of Transgenic Maize Events Via Agrobacterium

Agrobacterium tumefaciens harboring a binary donor vector containing a phosphomannose-isomerase selectable marker (PMI) in a promoter trap, and a reporter marker (dsRed or YFP) was streaked out from a −80° C. frozen aliquot onto solid PHI-L medium and cultured at 28° C. in the dark for 2-3 days. PHI-L media comprised 25 ml/L stock solution A, 25 ml/L stock solution B, 450.9 ml/L stock solution C and spectinomycin added to a concentration of 50 mg/L in sterile ddH2O (stock solution A: K2HPO4 60.0 g/L, NaH2PO4 20.0 g/L, adjust pH to 7.0 with KOH and autoclave; stock solution B: NH4C1 20.0 g/L, MgSO4-7H2O 6.0 g/L, KCl 3.0 g/L, CaCl₂) 0.20 g/L, FeSO4.7H2O 50.0 mg/L, autoclave; stock solution C: glucose 5.56 g/L, agar 16.67 g/L and autoclave). Agrobacterium to be used for transformation were grown on solid medium, and/or in liquid culture, as described below.

### Growing Agrobacterium on Solid Medium

A single colony or multiple colonies were picked from the master plate and streaked onto a plate containing PHI-M medium (yeast extract (Difco) 5.0 g/L; peptone (Difco) 10.0 g/L; NaCl 5.0 g/L; agar (Difco) 15.0 g/L; pH 6.8, containing 50 mg/L spectinomycin), and incubated at 28° C. in the dark for 1-2 days.

Five mL Agrobacterium infection medium (PHI-A: CHU(N6) basal salts (Sigma C-1416) 4.0 g/L, Eriksson's vitamin mix (1000×, Sigma-1511) 1.0 ml/L; thiamine-HCl 0.5 mg/L (Sigma); 2,4-dichlorophenoxyacetic acid (2,4-D, Sigma) 1.5 mg/L; L-proline (Sigma) 0.69 g/L; sucrose (Mallinckrodt) 68.5 g/L; glucose (Mallinckrodt) 36.0 g/L; pH 5.2; or, PHI-I: MS salts (GIBCO BRL) 4.3 g/L; nicotinic acid (Sigma) 0.5 mg/L; pyridoxine-HCl (Sigma) 0.5 mg/L; thiamine-HCl 1.0 mg/L; myo-inositol (Sigma) 0.10 g/L; vitamin assay casamino acids (Difco Lab) 1 g/L; 2, 4-D 1.5 mg/L; sucrose 68.50 g/L; glucose 36.0 g/L; adjust pH to 5.2 w/KOH and filter-sterilize) and 5 μL of 100 mM 3′-5′-dimethoxy-4′-hydroxyacetophenone (acetosyringone) were added to a 14 mL tube. About 3 full loops of Agrobacterium were suspended in the tube which was then vortexed to make an even suspension. One mL of the suspension was transferred to a spectrophotometer tube and the OD of the suspension was adjusted to 0.35-2.0 at 550 nm to yield an Agrobacterium concentration of about 0.5-2.0×109 cfu/mL. The final Agrobacterium suspension was aliquoted into 2 mL microcentrifuge tubes, each containing 1 mL of the suspension. The suspensions were then used for transformation as soon as possible.

### Growing Agrobacterium on Liquid Medium

One day before infection, a 125 mL flask was set up with 30 mL of 557A media (10.5 g/L potassium phosphate dibasic, 4.5 g/L potassium phosphate monobasic, 1.0 g/L ammonium sulfate, 0.5 g/L sodium citrate dihydrate, 0.2% (w/v) sucrose, 1 mM magnesium sulfate) with 30 μL each of spectinomycin (50 mg/mL) and acetosyringone (20 mg/mL). One-half loopful of Agrobacterium was suspended into each flask grown overnight at 28° C. with shaking at 200 rpm. The Agrobacterium culture was centrifuged at 5000 rpm for 10 min. The supernatant was removed and the Agrobacterium infection medium+acetosyringone solution was added. The bacteria were resuspended by vortexing and the OD of Agrobacterium suspension was adjusted to 0.35-2.0 at 550 nm.

### Maize Transformation

Ears of a maize (Zea mays L.) cultivar, PHR03, were surface-sterilized for 15-20 min in 20% (v/v) bleach (5.25% sodium hypochlorite) plus 1 drop of Tween 20 followed by 3 washes in sterile water. Immature embryos (IEs), typically 1.5-1.8 mm, were isolated from ears and were placed in 2 ml of the Agrobacterium infection medium+acetosyringone solution. The solution was drawn off and 1 ml of Agrobacterium suspension was added to the embryos, vortexed for 5-10 seconds, and then incubated 5 min at room temperature. The suspension of Agrobacterium and embryos were poured onto co-cultivation medium. Any embryos left in the tube were transferred to the plate using a sterile spatula. The Agrobacterium suspension was drawn off and the embryos placed axis side down on the media. The plate was sealed with PARAFILM™ tape and incubated in the dark at 21° C. for 1-3 days of co-cultivation.

Embryos were transferred to resting medium without selection. Three to 7 days later, they were transferred to green tissue induction medium (DBC3: 4.3 g/L MS salts, 30 g/L maltose, 1 mg/mL thiamine-HCl, 0.25 g/L myo-inositol, 1 g/L N-Z-amine-A (casein hydrolysate), 0.69 g/L proline, 4.9 μM CuSO4, 1.0 mg/L 2,4-D, 0.5 mg/L BAP; pH 5.8 3.5 g/L Phytagel) supplemented with mannose or other appropriate selective agent. Three weeks after the first round of selection, cultures were transferred to fresh green tissue induction medium containing a selective agent at 3- to 4-week intervals. Once transformed, transgenic green tissues are selected and cultured essentially as described in U.S. Pat. No. 7,102,056, and publication US20130055472, each of which is herein incorporated by reference in their entirety.

### Example 13: Generation of Target Lines for Agrobacterium SSI

A site-specific integration (SSI) target line was created in a maize cultivar, using Agrobacterium mediated immature embryo transformation essentially as described in U.S. Pat. No. 6,187,994, herein incorporated by reference in its entirety. A target site operably linked to a promoter trap is used to aid in target event identification, and SSI event identification. Lines comprising a promoter trap target site were generated by transformation with a construct comprising: PSA2-LOXP-UbiZMPro-FRT1-NptII::PinII+-FRT6.

### Example 14: Binary Vector Design for Agro-Mediated Site-Specific Integration in Plants

The binary vector design contains a Donor DNA flanked by heterologous FRT sites (FRT1/6), a FLP gene and the DevGene on the T-DNA delivered by Agro strain LBA4404 TD-Thy/PHP71539:
RB-OSActPro::WUS::IN2-1 TERM+UbiZMPro::BBM::OS-T28 TERM+UbiZMPro::FLP::PINII TERM-AT-T9 TERM+FRT1-PMI:PINII TERM-CZ19B1 TERM+ATTR4-CCDB-ATTR3+FRT6-LB.

Immature embryos with the target line are infected, and the SSI events are selected and characterized.

### Example 15: Promoter for Germline Excision

Three different maize specific germline promoters, RKD1, RKD2, and PG47 driving a Cre-recombinase gene were tested for excising marker genes in T1 events. RKD1 and RKD2 are ovule specific promoter, while PG47 is a pollen specific promoter and are expressed in the specific tissue-types.

### Example 16: Binary Vectors Design for Agro-Mediated Marker-Free Site-Specific Integration in Plants

The binary vector designs contain the Donor DNA plus an expression cassette containing the germline specific promoter driving a Cre-recombinase gene flanked by the 3′LOXP site placed downstream of the PMI::PINII TERM. The binary vectors designs (RKD1Pro::Cre), (RKD2:Cre), and (PG47::Cre) were delivered by an Agro strain:
RB-OSActPro::WUS::IN2-1 TERM+UbiZMPro::BBM::OS-T28 TERM+UbiZMPro::FLP::PINII TERM-AT-T9 TERM+FRT1-PMI:PINII TERM-CZ19B1 TERM+ZMRKD1::MO-CRE:SP-CP18 TERM+LOXP-ATTR4-CCDB-ATTR3+FRT6-LB
RB-OSActPro::WUS::IN2-1 TERM+UbiZMPro::BBM::OS-T28 TERM+UbiZMPro::FLP::PINII TERM-AT-T9 TERM+FRT1-PMI:PINII TERM-CZ19B1 TERM+ZMRKD2::MO-CRE: SP-CP18 TERM+LOXP-ATTR4-CCDB-ATTR3+FRT6-LB
RB-OSActPro::WUS::IN2-1 TERM+UbiZMPro::BBM::OS-T28 TERM+30 UbiZMPro::FLP::PINII TERM-AT-T9 TERM+FRT1-PMI:PINII TERM-CZ19B1 TERM+PG47Pro::MO-CRE:SP-CP18 TERM+LOXP-ATTR4-CCDB-ATTR3+FRT6-LB

Immature embryos with the target line were infected, and the SSI events were selected and characterized.

### Example 17: Testing of RKD1, RKD2 and PG47 Construct for Site-Specific Integration

Following retransformation of immature embryos containing the target with Agrobacterium strains containing the three binary vectors described above with insecticidal protein (IP) genes A and B, SSI events were selected on a media supplemented with mannose (PMI selection as described in U.S. Pat. Nos. 5,994,629 and 5,767,378 each of which is incorporated herein by reference in its entirety). Putative callus events were identified by culturing the retransformed embryos on media supplemented with mannose. Transformants wherein the target locus (NptII) was replaced with the polynucleotide construct (PMI/MO-CRE/IPs genes) were identified by their callus morphology. These events were regenerated and the TO plants were analyzed using standard qPCR assays. Table 14 shows the transformation frequency and frequency of site-specific recombination events recovered from maize inbred line HC69.

TABLE 14

| Trait cassette | Promoter: CRE | Embryo # | T0% | T0 QE % | UQE % |
|---|---|---|---|---|---|
| A | ZM-RKD1 | 405 | 21.5 | 44.7 | 9.6 |
| | ZM-RKD2 | 408 | 18.9 | 37.5 | 7.1 |
| | PG47 | 393 | 26.5 | 43.9 | 11.6 |
| B | RKD1 | 424 | 6.4 | 48.1 | 3.1 |
| | RKD2 | 410 | 5.9 | 54.2 | 3.2 |
| | PG47 | 416 | 13.2 | 63.6 | 8.4 |

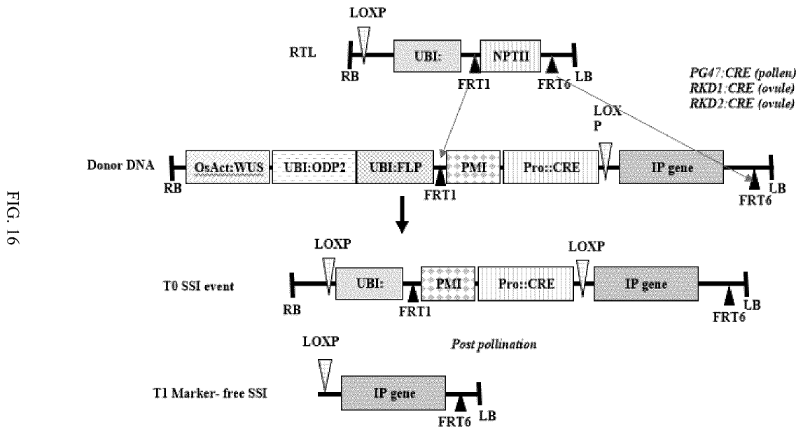

The process of generating marker-free SSI event generation is presented in FIG. 16. Once the TO SSI events are identified, these events are grown to maturity and pollinated with wild-type pollens, transgenic pollen or selfed to determine the excision efficiency with different germline promoters.

### Example 18: Confirmation of Marker-Gene Excision in T₁ Plants

Three T0 SSI events identified in Table 14 were grown to maturity and different pollination treatments, 1) self, 2) carry-in (wild-type pollen) and 3) carry-out (transgenic pollen to wild-type plant) were carried out to confirm excision. Post pollination, the T₁ seeds were germinated, sampled and standard PCR assay designed to detect the FRT junctions (FRT1 & FRT6) and copy number determination of PMI, MoCRE gene and IP gene (Trait gene) were applied as shown in Table 15 (Poll. type=Pollination type). The assays detected the excised events null for the PMI selectable marker and Mo-CRE genes flanked by LOXP sites and identified the trait gene only events (FIG. 16).

TABLE 15

| | | FRT6 | | Trait | FRT1 | | |
|---|---|---|---|---|---|---|---|
| | Excision | junction | MoCRE | gene | junction | PMI | Poll. |
| Pro:CRE | types | PCR | PCR | copy # | PCR | copy # | type |
| PG47 | 1 copy | POSITIVE | 1 | 2 | POSITIVE | 1 | self |
| | excised | | | | | | |
| PG47 | 1 copy | POSITIVE | 1 | 2 | POSITIVE | 1 | self |
| | excised | | | | | | |
| PG47 | hemi | POSITIVE | NULL | 1 | NEGATIVE | NULL | self |
| | excised | | | | | | |
| PG47 | hemi | POSITIVE | NULL | 1 | NEGATIVE | NULL | self |
| | excised | | | | | | |
| PG47 | hemi | POSITIVE | NULL | 1 | NEGATIVE | NULL | carry |
| | excised | | | | | | out |
| PG47 | hemi | POSITIVE | NULL | 1 | NEGATIVE | NULL | carry |
| | excised | | | | | | out |
| PG47 | hemi | POSITIVE | NULL | 1 | NEGATIVE | NULL | carry |
| | excised | | | | | | out |
| ZM- | 1 copy | POSITIVE | 1 | 2 | POSITIVE | 1 | self |
| RKD1 | excised | | | | | | |
| ZM- | hemi | POSITIVE | NULL | 1 | NEGATIVE | NULL | carry |
| RKD1 | excised | | | | | | in |
| ZM- | hemi | POSITIVE | NULL | 1 | NEGATIVE | NULL | carry |
| RKD1 | excised | | | | | | in |
| ZM- | hemi | POSITIVE | NULL | 1 | NEGATIVE | NULL | carry |
| RKD1 | excised | | | | | | in |
| ZM- | homo | POSITIVE | NULL | 2 | NEGATIVE | NULL | self |
| RKD2 | excised | | | | | | |
| ZM- | homo | POSITIVE | NULL | 2 | NEGATIVE | NULL | self |
| RKD2 | excised | | | | | | |
| ZM- | homo | POSITIVE | NULL | 2 | NEGATIVE | NULL | self |
| RKD2 | excised | | | | | | |
| ZM- | homo | POSITIVE | NULL | 2 | NEGATIVE | NULL | self |
| RKD2 | excised | | | | | | |
| ZM- | homo | POSITIVE | NULL | 2 | NEGATIVE | NULL | self |
| RKD2 | excised | | | | | | |
| ZM- | hemi | POSITIVE | NULL | 1 | NEGATIVE | NULL | carry |
| RKD2 | excised | | | | | | in |
| ZM- | hemi | POSITIVE | NULL | 1 | NEGATIVE | NULL | carry |
| RKD2 | excised | | | | | | in |
| ZM- | hemi | POSITIVE | NULL | 1 | NEGATIVE | NULL | carry |
| RKD2 | excised | | | | | | in |

### Example 19: Segregation of the Trait Genes in T₁ Progenies

The excised T₁ plants were sampled and quantitative PCR designed to detect the copy number of the trait gene was carried out to confirm the segregation of the trait genes in T₁ progenies. The segregation analysis confirmed the expected Mendalian inheritance of the trait genes in the different pollination types. All three promoters showed the typical 1:2:1 segregation for the trait gene in the T₁ progenies of the marker free events (Table 16 (TO Poll.=T0 Pollination; Seg. ratio=Segregation ratio)).

TABLE 16

| | | | # T1 | | | | | | | |
|---|---|---|---|---|---|---|---|---|---|---|
| | | | plants | | Homo | Homo | Hemi | | | |
| | | T0 | for | Homo- | 1 copy | non- | non- | Hemi | | Seg. |
| Trait | Pro:CRE | Poll. | PCR | excised | excised | excised | excised | excised | Nulls | ratio |
| A | PG47 | Self | 52 | | 14 | 2 | 14 | 10 | 12 | 1:2:1 |
| | | Carry | 17 | | | | 10 | | 7 | 1:1 |
| | | in | | | | | | | | |
| | | Carry | 46 | | | | 2 | 23 | 21 | 1:1 |
| | | out | | | | | | | | |
| | RKD1 | Self | 8 | | 1 | | 3 | 1 | 3 | 1:2:1 |
| | | Carry | 15 | | | | | 7 | 8 | 1:1 |
| | | in | | | | | | | | |
| | RKD2 | Self | 45 | 12 | 3 | | 4 | 19 | 7 | 1:2:1 |
| | | Carry | 38 | | | | 2 | 18 | 18 | 1:1 |
| | | in | | | | | | | | |
| B | PG47 | Self | 76 | | 9 | 5 | 21 | 14 | 27 | 1:2:1 |
| | | Carry | 45 | | | | 28 | | 17 | 1:1 |
| | | in | | | | | | | | |
| | | Carry | 29 | | | | 2 | 15 | 12 | 1:1 |
| | | out | | | | | | | | |
| | RKD1 | Self | 37 | 7 | 3 | 1 | 6 | 8 | 12 | 1:2:1 |
| | | carry | 32 | | | | | 15 | 17 | 1:1 |
| | | in | | | | | | | | |
| | RKD2 | Self | 51 | 10 | 1 | | 1 | 25 | 14 | 1:2:1 |
| | | Carry | 68 | | | | | 37 | 31 | 1:1 |
| | | in | | | | | | | | |

### Example 20: Other Approaches with CTBS

The sequences derived for the methods described below can be tested between two expression cassettes containing reporter genes. Expression analysis will be performed to evaluate the neighboring effects on expression characteristics for both gene cassettes in a gene stack configuration relative to single gene vectors and gene stacked vectors without a CTB/CIS sequence. Examples of experimental data from these approaches are shown under each category. These methods, in addition to those described above, are contemplated, including but not limited to the following.

### Chromatin Modification:

Gene expression networks are typically controlled by chromatin modifications. The elements in open chromatin will be determined and evaluated for CTB/CIS activity. The sequences were mined from a proprietary maize ATAC-Seq database or from the DNase Hypersensitivity (DHS) external source (Plant DHS database, plantdhs.org/). Transient assays to date with some of these sequences (ranging from 30 bp to 1 kb) showed CTB/CIS activity. Selected CTBs were also evaluated in stable corn plants in different tissues (Tables 17-21).

TABLE 17
Results from leaves of stably transformed maize plants

| SEQ ID NO | CTB | First Cassette | Second Cassette |
|---|---|---|---|
| | No CTB | 3 | 1 |
| 221 | DHS1 | 6 | 2 |
| 222 | DHS2 | 1 | No expression |
| 223 | DHS3 | 1 | 3 |
| 224 | DHS4 | 5 | 5 |
| 225 | DHS5 | 6 | 2 |
| 226 | DHS6 | 0 | 2 |
| 227 | DHS7 | 3 | 1 |
| 228 | DHS8 | 3 | 1 |
| 229 | DHS9 | 3 | 9 |
| 230 | DHS10 | 3 | 0 |

TABLE 18
Results from silk of stably transformed maize plants

| SEQ ID NO | CTB | First Cassette | Second Cassette |
|---|---|---|---|
| | No CTB | 3 | 2 |
| 221 | DHS1 | 6 | 5 |
| 222 | DHS2 | 8 | No expression |
| 223 | DHS3 | 8 | 3 |
| 224 | DHS4 | 7 | 5 |
| 225 | DHS5 | 7 | 3 |
| 226 | DHS6 | 5 | 6 |
| 227 | DHS7 | 4 | 4 |
| 228 | DHS8 | 6 | 5 |
| 229 | DHS9 | 6 | 4 |
| 230 | DHS10 | 4 | 6 |

TABLE 19
Results from stalk of stably transformed maize plants

| SEQ ID NO | CTB | First Cassette | Second Cassette |
|---|---|---|---|
| | No CTB | 7 | 5 |
| 221 | DHS1 | 4 | 5 |
| 222 | DHS2 | 1 | No expression |
| 223 | DHS3 | 8 | 5 |
| 224 | DHS4 | 9 | 9 |
| 225 | DHS5 | 6 | 6 |
| 226 | DHS6 | 9 | 6 |
| 227 | DHS7 | 7 | 9 |
| 228 | DHS8 | 7 | 8 |
| 229 | DHS9 | 5 | 4 |
| 230 | DHS10 | 6 | 5 |

TABLE 20
Results from husk of stably transformed maize plants

| | CTB | First Cassette | Second Cassette |
|---|---|---|---|
| | No CTB | 0 | 3 |
| 221 | DHS1 | 3 | 2 |
| 222 | DHS2 | 5 | No expression |
| 223 | DHS3 | 4 | 5 |
| 224 | DHS4 | 4 | 6 |
| 225 | DHS5 | 3 | 4 |
| 226 | DHS6 | 3 | 3 |
| 227 | DHS7 | 2 | 4 |
| 228 | DHS8 | 1 | 1 |
| 229 | DHS9 | 1 | 4 |
| 230 | DHS10 | 4 | 3 |

TABLE 21
Results from R1 leaf of stably transformed maize plants

| | CTB | First Cassette | Second Cassette |
|---|---|---|---|
| | No CTB | 8 | 3 |
| 221 | DHS1 | 7 | 5 |
| 222 | DHS2 | 4 | No expression |
| 223 | DHS3 | 3 | 5 |
| 224 | DHS4 | 2 | 6 |
| 225 | DHS5 | 4 | 6 |
| 226 | DHS6 | 9 | 5 |
| 227 | DHS7 | 1 | 8 |
| 228 | DHS8 | 2 | 8 |
| 229 | DHS9 | 5 | 6 |
| 230 | DHS10 | 6 | 4 |

0-9 scale based on the raw data first being normalized to the respective single gene control, then ranked based on the highest and lowest values.
0 = expression is 40%-49% of the single gene control
1 = expression is 50%-59% of the single gene control
2 = expression is 60%-69% of the single gene control
3 = expression is 70%-79% of the single gene control
4 = expression is 80%-89% of the single gene control
5 = expression is 90%-99% of the single gene control
6 = expression is 100%-109% of the single gene control
7 = expression is 110%-119% of the single gene control
8 = expression is 120%-129% of the single gene control
9 = expression is ≥130% of the single gene control

Insulator Signatures from Different Species:

Known insulator sequences from public data have been and will be used to identify orthologous signatures in a plant species of interest. For example, Miklos Gaszner et al. (1999) showed enhancer blocking activity from a 24 bp sequence of the Drosophila scs element. This sequence was used to identify homologous sequences from maize, Arabidopsis and soy. The homologous sequences vary from 15 bp to 50 bp. These sequences are being evaluated in 1× and 4× copies in transient assays for CTB/CIS performance (SEQ IDS 195 to 209). Some sequences show good activity (see Table 22).

TABLE 22
Transient maize results for orthologous
signatures of SCS binding sites

| | | First | Second |
|---|---|---|---|
| SEQ ID NO | CTB | Cassette | Cassette |
| | No CTB | 4 | 4 |
| 195 | 4x AT-CYT P450 | 2 | 4 |
| 196 | 4x AT-ICGP | 5 | 5 |
| 197 | 4x AT-ITGP | 3 | 4 |
| 198 | 4x AT-PHY A | 1 | 9 |
| 199 | 4x AT-RING | 4 | 4 |
| 200 | 4x ZM-IDGP | 2 | 4 |
| 201 | 4x ZM-ICGP | 8 | 9 |
| 202 | 4x ZM-IDGP-2 | 3 | 4 |
| 203 | 4x ZM-IDGP-3 | 2 | 4 |
| 204 | 4x ZM-IDGP-4 | 1 | 0 |
| 205 | 4x GM-CYT P450 | 4 | 8 |
| 206 | 4x GM-CYT P450-2 | 6 | 8 |
| 207 | 4x GM-IDGP CTB (REV) | 3 | 4 |
| 208 | 4x GM-IDGP | 4 | 5 |
| 209 | 4x GM-CYT P450-3 | 4 | 7 |

0-9 scale based on the raw data first being normalized to the respective single gene control, then ranked based on the highest and lowest values.
0 = expression is 40%-49% of the single gene control
1 = expression is 50%-59% of the single gene control
2 = expression is 60%-69% of the single gene control
3 = expression is 70%-79% of the single gene control
4 = expression is 80%-89% of the single gene control
5 = expression is 90%-99% of the single gene control
6 = expression is 100%-109% of the single gene control
7 = expression is 110%-119% of the single gene control
8 = expression is 120%-129% of the single gene control
9 = expression is ≥130% of the single gene control

Other approaches include the following:

A 2 kb fragment from petunia, TBS (transformation booster sequence, Jean-Michel Hily et al. 2009), was analyzed for homologous sequences from different plant species and fragments ranging from 15 bp to 300 bp fragments are being evaluated (SEQ IDs 210 to 220).

CTCF (CCCTC-Binding factor) is involved in many cellular processes, including transcriptional regulation, insulator activity, V(D)J recombination and regulation of chromatin architecture.

CTCF-like motifs have been identified from Arabidopsis, soy and maize and will be evaluated in 2× to 4× copies (SEQ IDs 165 to 194) for insulator or CTB like activity. The length of the individual sequences vary from 15 bp to 30 bp. Transient evaluation of 14 sequences in 4× copies is shown in Table 23.

TABLE 23
Maize results for orthologous CTCF motif sequences

| SEQ ID NO | CTB | First Cassette | Second Cassette |
|---|---|---|---|
| | No CTB | very low expression | 0 |
| 166 | 4X ZM-CTCF-1 | 0 | 1 |
| 168 | 4X ZM-CTCF-2 | 1 | 1 |
| 170 | 4X ZM-CTCF-3 | 2 | 2 |
| 172 | 4X ZM-CTCF-4 | 5 | 4 |
| 174 | 4X ZM-CTCF-5 | 2 | 4 |
| 176 | 4X AT-DUF626 | 2 | 6 |
| 178 | 4X AT-PCD | very low expression | 9 |
| 180 | 4X AT-DZFP | 3 | 3 |
| 182 | 4X AT-SAM | 1 | 1 |
| 184 | 4X AT-DUF936 | 2 | 2 |
| 186 | 4X GM-CTCF-1 | 0 | 1 |
| 188 | 4X GM-CTCF-2 | 0 | 0 |
| 190 | 4X GM-MAT-1 | 4 | 5 |
| 192 | 4X GM-CTCF-3 | 0 | 1 |
| 194 | 4X GM-MAT-2 | 1 | 1 |

0-9 scale based on the raw data first being normalized to the respective single gene control, then ranked based on the highest and lowest values.
0 = expression is 40%-49% of the single gene control
1 = expression is 50%-59% of the single gene control
2 = expression is 60%-69% of the single gene control
3 = expression is 70%-79% of the single gene control
4 = expression is 80%-89% of the single gene control
5 = expression is 90%-99% of the single gene control
6 = expression is 100%-109% of the single gene control
7 = expression is 110%-119% of the single gene control
8 = expression is 120%-129% of the single gene control
9 = expression is ≥130% of the single gene control

### DNA/Nucleosome Modification (Epigenetic Control):

Modifications to DNA, histone, and non-histone chromosomal proteins establish a complex regulatory network that controls genome function. Chemical modifications of histones include methylation, acetylation, phosphorylation, ubiquitination, and sumoylation. These properties will be leveraged to identify or design sequences for gene regulation. For example, the property of DNA methylation, in switching gene regulation, will be leveraged to alter the properties of the DNA sequence (CTB) positioned between two neighboring genes. Experiments in progress include testing a DNA fragment predicted to be methylated. Preliminary results indicate it has CTB/CIS activity when placed between two expression cassettes in a gene stack configuration, further evaluations in progress (SEQ ID 267).

### Terminators in 2× to 4× Copies:

Terminator sequences constitute the 3′ UTR or a combination of 3′ UTR and downstream sequences of up to 1 kb. Two to four terminators can be added together to build a CTB/CIS sequence to evaluate the impact on expression characteristics of both upstream and downstream cassettes in a transgenic plant. Work using this concept has been done in rice and maize. Some of the combinations include up to 4 terminators (Table 24) showed preserved expression characteristics when placed between neighboring genes in stable rice plants (Table 25).

### Intergenic Regions Between Dense Gene Pairs:

DNA sequence between highly or equally expressed gene pairs can display CTB/CIS activity. This intergenic region, which may include the 3′ UTR, can be up to 3 kb in length. Features in the intergenic region allow the native gene pair their expression characteristics. Isolation and insertion of these types of sequences, for example, in a stacked, transgene configuration may allow for the preservation of cassette expression characteristics, as if the cassettes were independent of each other. A combination of sequences from convergent gene pairs (Table 24) showed preserved expression patterns of neighboring transgenes (Table 25). Sequences from a variety of different plant species are being isolated.

### Transcriptional Termination Signals:

Termination signal sequences, which include poly(A) addition signals, are being evaluated. Poly(A) signal strength and/or clustering of poly(A) addition sites may contribute attributes to a sequence for enhanced CTB/CIS activity. Synthetic elements can be created by combining learnings from experiments currently in progress. A set of completed experiments has tested a synthetic sequence consisting of poly(A) signal sequences from 5 terminators combined together (Table 24). Irrespective of direction, some of the combinations showed CTB/CIS activity (Table 25).

TABLE 24
Terminators, convergent gene pairs and
poly A signature sequences as CTBs

| SEQ | Vector | |
|---|---|---|
| ID NO | name/CTB | Description |
| | STV 2 | No CTB |
| | STV4 | Reporter gene1 |
| | STV5 | Reporter gene2 |
| 251 | STV6 | 3 kb coding sequence as CTB test |
| 252 | STV7 | 1.7 Kb coding sequence as CTB test |
| 253 | STV8 | Intergenic region and 3¹ UTR from Convergent gene |
| | | pair as CTB |
| 254 | STV9 | 4x terminator as CTB |
| 255 | STV10 | 4x terminator in reverse as CTB |
| 256 | STV11 | Intergenic region and 3¹ UTR from Convergent gene |
| | | pair as CTB |
| 257 | STV12 | 4x terminator as CTB |
| 258 | STV13 | 4x terminator in reverse as CTB test |
| 259 | STV14 | Intergenic region and 3¹ UTR from Convergent gene |
| | | pair as CTB |
| 260 | STV15 | 4x terminator as CTB |
| 261 | STV16 | 4x terminator in reverse as CTB test |
| 262 | STV17 | 1 Kb coding sequence as CTB test |
| 263 | STV18 | Poly A signal sequences from 5 Terminators as CTB |
| 264 | STV19 | Poly A signal sequences from 5 Terminators in |
| | | reverse as CTB test |
| 265 | STV20 | Poly A signal sequences from 5 Terminators as CTB |
| 266 | STV21 | Poly A signal sequences from 5 Terminators in |
| | | reverse as CTB test |

TABLE 25
Evaluation of Terminators, convergent gene pairs
and poly A signal sequences as CTBs in rice
stable plants. Results are from leaf tissue

| | Reporter gene 1 | Reporter gene 2 |
|---|---|---|
| | expression (%) | expression (%) |
| Vector | (upsteam cassette) | (downstream cassette) |
| STV4 | 100 | |
| STV5 | | 100 |
| STV2 | 144 | 99 |
| STV6 | 112 | 68 |
| STV7 | 122 | 94 |
| STV8 | 188 | 100 |
| STV9 | 207 | 197 |
| STV10 | 157 | 85 |
| STV11 | 286 | 192 |
| STV12 | 177 | 112 |
| STV13 | 300 | 53 |
| STV14 | 202 | 124 |
| STV15 | 206 | 154 |
| STV16 | 1 | 0.3 |
| STV17 | 277 | 126 |
| STV18 | 157 | 167 |
| STV19 | 165 | 175 |
| STV20 | 383 | 63 |
| STV21 | 337 | 533 |

CTB/CIS Regions Upstream of, or within, Promoters

Regulatory regions in promoters or 5′ flanking regions of genes can have CTB/CIS activity. These may function by binding protein, bending nucleic acids or a combination of both, thereby limiting the effect of one expression cassette on another in a plant or plant cell. Several candidates have been tested. Examples include a segment from the Sb-Gly promoter and another from the OEBF promoter (Seq ID 243 to 250). They work as duplicated copies and in combination (one Sb-GLY and one Zm-OEBF).

### Library (Genomic DNA Fragments):

A library of genomic DNA (fragments of 300 bp to 2 kb) from different plant species can be cloned between 2 genes and evaluated for CTB/CIS activity. Source material can be broad, but currently a STAR-seq library exists and sequences that provide no or limited expression enhancement can be evaluated for CTB/CIS activity.

### Example 21: Sequences

The sequence descriptions and sequence listing attached hereto comply with the rules governing nucleotide and amino acid sequence disclosures in patent applications as set forth in 37 C.F.R. §§ 1.831 through 1.835. The sequence descriptions comprise the three letter codes for amino acids as defined in 37 C.F.R. §§ 1.831 through 1.835, which are incorporated herein by reference. Variable nucleotides are indicated as: U=T # alias Uracil to Thymine (permit U in input sequences); R=AG; Y=CT; K=GT; M=AC; S=CG; W=AT; B=CGT; D=GAT; H=ACT; V=ACG; N=ACGT # wildcard symbol.

See Table 26 for sequences useful in the present disclosure.

TABLE 26
Sequence Table

| | Polynucleotide | | |
|---|---|---|---|
| SEQ | (DNA) or | | |
| ID | Polypeptide | | |
| NO: | (PRT) | NAME | DESCRIPTION |
| 1 | DNA | ZM-T1S1C1 CTB | Zea Mays DNA sequence |
| | | | ZM-T1S1C1 CTB |
| 2 | DNA | ZM-T1S2C3 CTB | Zea Mays DNA sequence |
| | | | ZM-T1S2C3 CTB |
| 3 | DNA | ZM-T1S2C3-2 CTB | Zea Mays DNA sequence |
| | | | ZM-T1S2C3-2 CTB |
| 4 | DNA | ZM-T1S2C6 CTB | Zea Mays DNA sequence |
| | | | ZM-T1S2C6 CTB |
| 5 | DNA | ZM-T1S2C7 CTB | Zea Mays DNA sequence |
| | | | ZM-T1S2C7 CTB |
| 6 | DNA | ZM-T1S2C8 CTB | Zea Mays DNA sequence |
| | | | ZM-T1S2C8 CTB |
| 7 | DNA | ZM-T1S2C9 CTB | Zea Mays DNA sequence |
| | | | ZM-T1S2C9 CTB |
| 8 | DNA | ZM-T1S2C9-2 CTB | Zea Mays DNA sequence |
| | | | ZM-T1S2C9-2 CTB |
| 9 | DNA | ZM-T1S2C1 CTB | Zea Mays DNA sequence |
| | | | ZM-T1S2C1 CTB |
| 10 | DNA | ZM-T1S6C6 CTB | Zea Mays DNA sequence |
| | | | ZM-T1S6C6 CTB |
| 11 | DNA | ZM-T1S6C1 CTB | Zea Mays DNA sequence |
| | | | ZM-T1S6C1 CTB |
| 12 | DNA | ZM-T2S1C1 CTB | Zea Mays DNA sequence |
| | | | ZM-T2S1C1 CTB |
| 13 | DNA | ZM-T2S1C3 CTB | Zea Mays DNA sequence |
| | | | ZM-T2S1C3 CTB |
| 14 | DNA | ZM-T2S1C4 CTB | Zea Mays DNA sequence |
| | | | ZM-T2S1C4 CTB |
| 15 | DNA | ZM-T2S1C5 CTB | Zea Mays DNA sequence |
| | | | ZM-T2S1C5 CTB |
| 16 | DNA | ZM-T2S2C8 CTB | Zea Mays DNA sequence |
| | | | ZM-T2S2C8 CTB |
| 17 | DNA | ZM-T2S2C9 CTB | Zea Mays DNA sequence |
| | | | ZM-T2S2C9 CTB |
| 18 | DNA | ZM-T2S2C4 CTB | Zea Mays DNA sequence |
| | | | ZM-T2S2C4 CTB |
| 19 | DNA | ZM-T2S2C4-2 CTB | Zea Mays DNA sequence |
| | | | ZM-T2S2C4-2 CTB |
| 20 | DNA | ZM-T2S2C2 CTB | Zea Mays DNA sequence |
| | | | ZM-T2S2C2 CTB |
| 21 | DNA | ZM-T2S2C3 CTB | Zea Mays DNA sequence |
| | | | ZM-T2S2C3 CTB |
| 22 | DNA | ZM-T2S2C2-2 CTB | Zea Mays DNA sequence |
| | | | ZM-T2S2C2-2 CTB |
| 23 | DNA | ZM-T2S2C2-3 CTB | Zea Mays DNA sequence |
| | | | ZM-T2S2C2-3 CTB |
| 24 | DNA | ZM-T2S2C2-4 CTB | Zea Mays DNA sequence |
| | | | ZM-T2S2C2-4 CTB |
| 25 | DNA | ZM-T2S2C7 CTB | Zea Mays DNA sequence |
| | | | ZM-T2S2C7 CTB |
| 26 | DNA | ZM-T2S2C5 CTB | Zea Mays DNA sequence |
| | | | ZM-T2S2C5 CTB |
| 27 | DNA | ZM-T2S2C5-2 CTB | Zea Mays DNA sequence |
| | | | ZM-T2S2C5-2 CTB |
| 28 | DNA | ZM-T2S2C5-3 CTB | Zea Mays DNA sequence |
| | | | ZM-T2S2C5-3 CTB |
| 29 | DNA | ZM-T2S2C5-4 CTB | Zea Mays DNA sequence |
| | | | ZM-T2S2C5-4 CTB |
| 30 | DNA | ZM-T1S1C5 CTB | Zea Mays DNA sequence |
| | | | ZM-T1S1C5 CTB |
| 31 | DNA | ZM-T1S6C5 CTB | Zea Mays DNA sequence |
| | | | ZM-T1S6C5 CTB |
| 32 | DNA | ZM-T2S1C5-2 CTB | Zea Mays DNA sequence |
| | | | ZM-T2S1C5-2 CTB |
| 33 | DNA | ZM-T2S1C5-3 CTB | Zea Mays DNA sequence |
| | | | ZM-T2S1C5-3 CTB |
| 34 | DNA | ZM-T1S6C5-2 CTB | Zea Mays DNA sequence |
| | | | ZM-T1S6C5-2 CTB |
| 35 | DNA | ZM-T2S2C5-5 CTB | Zea Mays DNA sequence |
| | | | ZM-T2S2C5-5 CTB |
| 36 | DNA | INS1 | Arabidopsis thaliana DNA sequence Cross-talk blocker |
| | | | sequence INS1 from region upstream of AT-4G15300 |
| 37 | DNA | INS2 | Arabidopsis thaliana DNA sequence Cross-talk blocker |
| | | | sequence INS2 from region upstream of AT-4G15300 |
| 38 | DNA | INS3 | Arabidopsis thaliana DNA sequence Cross-talk blocker |
| | | | sequence INS3 from region upstream of AT-4G15300 |
| 39 | DNA | INS4 | Arabidopsis thaliana DNA sequence Cross-talk blocker |
| | | | sequence INS4 from region upstream of AT-4G15300 |
| 40 | DNA | INS5 | Arabidopsis thaliana DNA sequence Cross-talk blocker |
| | | | sequence INS5 from region upstream of AT-4G15280 |
| 41 | DNA | INS6 | Arabidopsis thaliana DNA sequence Cross-talk blocker |
| | | | sequence INS6 from region upstream of AT-4G15280 |
| 42 | DNA | INS7 | Arabidopsis thaliana DNA sequence Cross-talk blocker |
| | | | sequence INS7 from region upstream of AT-4G15280 |
| 43 | DNA | INS8 | Arabidopsis thaliana DNA sequence Cross-talk blocker |
| | | | sequence INS8 from region upstream of AT-4G15280 |
| 44 | DNA | INS9 | Arabidopsis thaliana DNA sequence Cross-talk blocker |
| | | | sequence INS9 from region upstream of AT-1G60140 |
| 45 | DNA | INS10 | Arabidopsis thaliana DNA sequence Cross-talk blocker |
| | | | sequence INS10 from region upstream of AT-1G60140 |
| 46 | DNA | INS11 | Arabidopsis thaliana DNA sequence Cross-talk blocker |
| | | | sequence INS11 from region upstream of AT-1G60140 |
| 47 | DNA | INS12 | Arabidopsis thaliana DNA sequence Cross-talk blocker |
| | | | sequence INS12 from region upstream of AT-1G60140 |
| 48 | DNA | INS24 | Arabidopsis thaliana DNA sequence Cross-talk blocker |
| | | | sequence INS24 from region upstream of AT-4G15290 |
| 49 | DNA | INS25 | Arabidopsis thaliana DNA sequence Cross-talk blocker |
| | | | sequence INS25 from region upstream of AT-4G15290 |
| 50 | DNA | INS26 | Arabidopsis thaliana DNA sequence Cross-talk blocker |
| | | | sequence INS26 from region upstream of AT-4G15290 |
| 51 | DNA | INS27 | Arabidopsis thaliana DNA sequence Cross-talk blocker |
| | | | sequence INS27 from region upstream of AT-4G15290 |
| 52 | DNA | INS13 | Artificial DNA sequence Cross-talk blocker sequence |
| | | (AT-5-IV-8) | INS13 comprising 5 motifs from AT-5-IV-7 |
| 53 | DNA | AT-5-IV-2 INS | Arabidopsis thaliana DNA sequence AT-5-IV-2 INS |
| 54 | DNA | AT-5-IV-7 INS | Arabidopsis thaliana DNA sequence AT-5-IV-7 INS |
| 55 | DNA | AT-14-II-2 INS | Arabidopsis thaliana DNA sequence AT-14-II-2 INS |
| 56 | DNA | AT-5-IV-2 INS | Arabidopsis thaliana DNA sequence AT-5-IV-2 INS |
| | | (DEL1) | (DEL1) |
| 57 | DNA | AT-5-IV-2 INS | Arabidopsis thaliana DNA sequence AT-5-IV-2 INS |
| | | (DEL2) | (DEL2) |
| 59 | DNA | AT-5-IV-2 INS | Arabidopsis thaliana DNA sequence AT-5-IV-2 INS |
| | | (DEL3) | (DEL3) |
| 59 | DNA | AT-5-IV-2 INS | Arabidopsis thaliana DNA sequence AT-5-IV-2 INS |
| | | (DEL4) | (DEL4) |
| 60 | DNA | AT-5-IV-2 INS | Arabidopsis thaliana DNA sequence AT-5-IV-2 INS |
| | | (DEL5) | (DEL5) |
| 61 | DNA | AT-5-IV-2 INS | Arabidopsis thaliana DNA sequence AT-5-IV-2 INS |
| | | (DEL6)) | (DEL6) |
| 62 | DNA | AT-5-IV-2 (DEL7) | Arabidopsis thaliana DNA sequence AT-5-IV-2 (DEL7) |
| 63 | DNA | AT-5-IV-2 (DEL8) | Arabidopsis thaliana DNA sequence AT-5-IV-2 (DEL8) |
| 64 | DNA | AT-5-IV-7A | Arabidopsis thaliana DNA sequence AT-5-IV-7A |
| 65 | DNA | AT-5-IV-7B | Arabidopsis thaliana DNA sequence AT-5-IV-7B |
| 66 | DNA | AT-5-IV-7C | Arabidopsis thaliana DNA sequence AT-5-IV-7C |
| 67 | DNA | EE3334 (COMP) | Arabidopsis thaliana DNA sequence EE3334 (COMP) |
| 68 | DNA | AT-5-IV-7D | Arabidopsis thaliana DNA sequence AT-5-IV-7D |
| 69 | DNA | ZM_CTB_T1S1C5_35 | Zea Mays DNA sequence ZM_CTB_T1S1C5_35 |
| 70 | DNA | ZM_CTB_T1S1C7_36 | Zea Mays DNA sequence ZM_CTB_T1S1C7_36 |
| 71 | DNA | ZM_CTB_T1S1C2_38 | Zea Mays DNA sequence ZM_CTB_T1S1C7_37 |
| 72 | DNA | ZM_CTB_T1S1C2_38 | Zea Mays DNA sequence ZM_CTB_T1S1C2_38 |
| 73 | DNA | ZM_CTB_T1S1C7_39 | Zea Mays DNA sequence ZM_CTB_T1S1C7_39 |
| 74 | DNA | ZM_CTB_T1S1C9_40 | Zea Mays DNA sequence ZM_CTB_T1S1C9_40 |
| 75 | DNA | ZM_CTB_T1S2C7_41 | Zea Mays DNA sequence ZM_CTB_T1S2C7_41 |
| 76 | DNA | ZM_CTB_T1S2C6_42 | Zea Mays DNA sequence ZM_CTB_T1S2C6_42 |
| 77 | DNA | ZM_CTB_T1S2C4_43 | Zea Mays DNA sequence ZM_CTB_T1S2C4_43 |
| 78 | DNA | ZM_CTB_T1S2C5_44 | Zea Mays DNA sequence ZM_CTB_T1S2C5_44 |
| 79 | DNA | ZM_CTB_T1S2C6_45 | Zea Mays DNA sequence ZM_CTB_T1S2C6_45 |
| 80 | DNA | ZM_CTB_T1S2C1_46 | Zea Mays DNA sequence ZM_CTB_T1S2C1_46 |
| 81 | DNA | ZM_CTB_T1S2C10_47 | Zea Mays DNA sequence ZM_CTB_T1S2C10_47 |
| 82 | DNA | ZM_CTB_T1S2C5_48 | Zea Mays DNA sequence ZM_CTB_T1S2C5_48 |
| 83 | DNA | ZM_CTB_T1S2C1_49 | Zea Mays DNA sequence ZM_CTB_T1S2C1_49 |
| 84 | DNA | ZM_CTB_T1S3C8_50 | Zea Mays DNA sequence ZM_CTB_T1S3C8_50 |
| 85 | DNA | ZM_CTB_T1S3C1_51 | Zea Mays DNA sequence ZM_CTB_T1S3C1_51 |
| 86 | DNA | ZM_CTB_T1S3C1_52 | Zea Mays DNA sequence ZM_CTB_T1S3C1_52 |
| 87 | DNA | ZM_CTB_T1S3C5_53 | Zea Mays DNA sequence ZM_CTB_T1S3C5_53 |
| 88 | DNA | ZM_CTB_T1S3C1_54 | Zea Mays DNA sequence ZM_CTB_T1S3C1_54 |
| 89 | DNA | ZM_CTB_T1S3C1_55 | Zea Mays DNA sequence ZM_CTB_T1S3C1_55 |
| 91 | DNA | ZM_CTB_T1S3C3_56 | Zea Mays DNA sequence ZM_CTB_T1S3C3_56 |
| 91 | DNA | ZM_CTB_T1S3C10_57 | Zea Mays DNA sequence ZM_CTB_T1S3C10_57 |
| 92 | DNA | ZM_CTB_T1S3C5_58 | Zea Mays DNA sequence ZM_CTB_T1S3C5_58 |
| 93 | DNA | ZM_CTB_T1S3C3_59 | Zea Mays DNA sequence ZM_CTB_T1S3C3_59 |
| 94 | DNA | ZM_CTB_T1S5C9_60 | Zea Mays DNA sequence ZM_CTB_T1S5C9_60 |
| 95 | DNA | ZM_CTB_T1S5C7_61 | Zea Mays DNA sequence ZM_CTB_T1S5C7_61 |
| 96 | DNA | ZM_CTB_T1S6C3_62 | Zea Mays DNA sequence ZM_CTB_T1S6C3_62 |
| 97 | DNA | ZM_CTB_T1S6C2_63 | Zea Mays DNA sequence ZM_CTB_T1S6C2_63 |
| 98 | DNA | ZM_CTB_T1S6C7_64 | Zea Mays DNA sequence ZM_CTB_T1S6C7_64 |
| 99 | DNA | ZM_CTB_T1S6C1_66 | Zea Mays DNA sequence ZM_CTB_T1S6C1_66 |
| 100 | DNA | ZM_CTB_T1S6C8_67 | Zea Mays DNA sequence ZM_CTB_T1S6C8_67 |
| 101 | DNA | ZM_CTB_T1S6C6_68 | Zea Mays DNA sequence ZM_CTB_T1S6C6_68 |
| 102 | DNA | ZM_CTB_T1S6C8_69 | Zea Mays DNA sequence ZM_CTB_T1S6C8_69 |
| 103 | DNA | ZM_CTB_T2S1C1_70 | Zea Mays DNA sequence ZM_CTB_T2S1C1_70 |
| 104 | DNA | ZM_CTB_T2S1C9_71 | Zea Mays DNA sequence ZM_CTB_T2S1C9_71 |
| 105 | DNA | ZM_CTB_T2S1C5_72 | Zea Mays DNA sequence ZM_CTB_T2S1C5_72 |
| 106 | DNA | ZM_CTB_T2S1C9_73 | Zea Mays DNA sequence ZM_CTB_T2S1C9_73 |
| 107 | DNA | ZM_CTB_T2S1C2_74 | Zea Mays DNA sequence ZM_CTB_T2S1C2_74 |
| 108 | DNA | ZM_CTB_T2S1C1_75 | Zea Mays DNA sequence ZM_CTB_T2S1C1_75 |
| 109 | DNA | ZM_CTB_T2S1C1_77 | Zea Mays DNA sequence ZM_CTB_T2S1C1_77 |
| 110 | DNA | ZM_CTB_T2S1C6_78 | Zea Mays DNA sequence ZM_CTB_T2S1C6_78 |
| 111 | DNA | ZM_CTB_T2S1C6_79 | Zea Mays DNA sequence ZM_CTB_T2S1C6_79 |
| 112 | DNA | ZM_CTB_T2S1C3_80 | Zea Mays DNA sequence ZM_CTB_T2S1C3_80 |
| 113 | DNA | ZM_CTB_T2S1C1_81 | Zea Mays DNA sequence ZM_CTB_T2S1C1_81 |
| 114 | DNA | ZM_CTB_T2S1C1_82 | Zea Mays DNA sequence ZM_CTB_T2S1C1_82 |
| 115 | DNA | ZM_CTB_T2S1C2_83 | Zea Mays DNA sequence ZM_CTB_T2S1C2_83 |
| 116 | DNA | ZM_CTB_T2S1C1_84 | Zea Mays DNA sequence ZM_CTB_T2S1C1_84 |
| 117 | DNA | ZM_CTB_T2S2C9_85 | Zea Mays DNA sequence ZM_CTB_T2S2C9_85 |
| 118 | DNA | ZM_CTB_T2S2C9_86 | Zea Mays DNA sequence ZM_CTB_T2S2C9_86 |
| 119 | DNA | ZM_CTB_T2S2C4_87 | Zea Mays DNA sequence ZM_CTB_T2S2C4_87 |
| 120 | DNA | ZM_CTB_T2S2C7_88 | Zea Mays DNA sequence ZM_CTB_T2S2C7_88 |
| 120 | DNA | ZM_CTB_T2S2C5_89 | Zea Mays DNA sequence ZM_CTB_T2S2C5_89 |
| 122 | DNA | ZM_CTB_T2S2C4_90 | Zea Mays DNA sequence ZM_CTB_T2S2C4_90 |
| 123 | DNA | ZM_CTB_T2S2C1_91 | Zea Mays DNA sequence ZM_CTB_T2S2C1_91 |
| 124 | DNA | ZM_CTB_T2S2C8_92 | Zea Mays DNA sequence ZM_CTB_T2S2C8_92 |
| 125 | DNA | ZM_CTB_T2S2C1_93 | Zea Mays DNA sequence ZM_CTB_T2S2C1_93 |
| 126 | DNA | ZM_CTB_T2S2C5_94 | Zea Mays DNA sequence ZM_CTB_T2S2C5_94 |
| 127 | DNA | ZM_CTB_T2S2C1_95 | Zea Mays DNA sequence ZM_CTB_T2S2C1_95 |
| 128 | DNA | ZM_CTB_T2S2C9_97 | Zea Mays DNA sequence ZM_CTB_T2S2C9_97 |
| 129 | DNA | ZM_CTB_T2S2C5_98 | Zea Mays DNA sequence ZM_CTB_T2S2C5_98 |
| 130 | DNA | ZM_CTB_T2S2C7_99 | Zea Mays DNA sequence ZM_CTB_T2S2C7_99 |
| 131 | DNA | ZM_CTB_T2S2C1_100 | Zea Mays DNA sequence ZM_CTB_T2S2C1_100 |
| 132 | DNA | AT-5-III-1 | Arabidopsis DNA sequence |
| | | | AT-5-III-1 |
| 133 | DNA | Motif Sequence 1 | Artificial DNA sequence |
| | | | Motif Sequence 1 |
| 134 | DNA | Motif Sequence 2 | Artificial DNA sequence |
| | | | Motif Sequence 2 |
| 135 | DNA | Motif Sequence 4 | Artificial DNA sequence |
| | | | Motif Sequence 4 |
| 136 | DNA | Motif Sequence 5 | Artificial DNA sequence |
| | | | Motif Sequence 5 |
| 137 | DNA | Motif Sequence 6 | Artificial DNA sequence |
| | | | Motif Sequence 6 |
| 138 | DNA | Motif Sequence 7 | Artificial DNA sequence |
| | | | Motif Sequence 7 |
| 139 | DNA | Motif Sequence 10 | Artificial DNA sequence |
| | | | Motif Sequence 10 |
| 140 | DNA | Motif Sequence 11 | Artificial DNA sequence |
| | | | Motif Sequence 11 |
| 141 | DNA | Motif Sequence 18 | Artificial DNA sequence |
| | | | Motif Sequence 18 |
| 142 | DNA | CTB vector depicted | Artificial DNA sequence for the CTB vector depicted in |
| | | in FIG. 5 | FIG. 5 |
| 143 | DNA | CTB vector depicted | Artificial DNA sequence for the CTB vector depicted in |
| | | in FIG. 6 | FIG. 6 |
| 144 | DNA | CTB vector depicted | Artificial DNA sequence for the CTB vector depicted in |
| | | in FIG. 7 | FIG. 7 |
| 145 | DNA | PHP96034 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + AT-5-IV-2 INS + NOS PRO::ZM- |
| | | | WUS2::IN2 TERM + FMV ENHANCER + PCSV ENH + |
| | | | MMV ENH + UBI1ZM PRO::UBI1ZM 5UTR::UBI1ZM |
| | | | INTRON1::ZM-ODP2::OS-T28 TERM + LOXP + SB-UBI |
| | | | PRO::SB-UBI INTRON1::ZSGREEN1::OS-UBI TERM + |
| | | | SB-ALS PRO::ZM-ALS::SB-UBI TERM + LB |
| 146 | DNA | PHP101513 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + ZM-T1S1C1 CTB + NOS |
| | | | PRO::ZM-WUS2::IN2 TERM + FMV ENHANCER + |
| | | | PCSV ENH + MMV ENH + UBI1ZM PRO::UBI1ZM |
| | | | 5UTR::UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 147 | DNA | PHP101514 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + ZM- T1S2C3 CTB + NOS |
| | | | PRO::ZM-WUS2::IN2 TERM + FMV ENHANCER + |
| | | | PCSV ENH + MMV ENH + UBI1ZM PRO::UBI1ZM |
| | | | 5UTR::UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 148 | DNA | PHP101652 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + ZM- T1S2C8 CTB + NOS |
| | | | PRO::ZM-WUS2::IN2 TERM + FMV ENHANCER + |
| | | | PCSV ENH + MMV ENH + UBI1ZM PRO::UBI1ZM |
| | | | 5UTR::UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 149 | DNA | PHP101653 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + ZM- T1S2C9 CTB + NOS |
| | | | PRO::ZM-WUS2::IN2 TERM + FMV ENHANCER + |
| | | | PCSV ENH + MMV ENH + UBI1ZM PRO::UBI1ZM |
| | | | 5UTR::UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 150 | DNA | PHP101654 | RB + LOXP + AT-5-IV-2 INS + ZM-HSP17.7 PRO::MO- |
| | | | CRE EXON1::ST-LS1 INTRON2::MO-CRE |
| | | | EXON2::PINII TERM + ZM- T2S2C9 CTB + NOS |
| | | | PRO::ZM-WUS2::IN2 TERM + FMV ENHANCER + |
| | | | PCSV ENH + MMV ENH + UBI1ZM PRO::UBI1ZM |
| | | | 5UTR::UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 151 | DNA | PHP97883 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-HSP17.7 PRO::MO-CRE |
| | | | EXON1::ST-LS1 INTRON2::MO-CRE EXON2::PINII |
| | | | TERM + LOXP + SB-UBI PRO::SB-UBI INTRON1:: |
| | | | ZSGREEN1::OS-UBI TERM + SB-ALS PRO::ZM- |
| | | | ALS::SB-UBI TERM + LB |
| 152 | DNA | PHP96037 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-2 INS + ZM-HSP17.7 |
| | | | PRO::MO-CRE EXON1::ST-LS1 INTRON2::MO-CRE |
| | | | EXON2::PINII TERM + AT-5-IV-2 INS + LOXP + SB- |
| | | | UBI PRO::SB-UBI INTRON1::ZSGREEN1::OS-UBI |
| | | | TERM + SB-ALS PRO::ZM-ALS::SB-UBI TERM + LB |
| 153 | DNA | PHP101595 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15300-I INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 154 | DNA | PHP101271 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15300-II INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 155 | DNA | PHP101811 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15300-IV INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 156 | DNA | PHP101272 | RB + LOXP + NOS PRO: ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15280-II INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 157 | DNA | PHP101596 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-1G60140-I INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 158 | DNA | PHP101512 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15290-I INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 159 | DNA | PHP101597 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + AT-4G15290-IV INS + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 160 | DNA | PHP101308 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-T2S2C8 CTB + ZM-HSP17.7 |
| | | | PRO::MO-CRE EXON1::ST-LS1 INTRON2::MO-CRE |
| | | | EXON2::PINII TERM + LOXP + SB-UBI PRO::SB-UBI |
| | | | INTRON1::ZSGREEN1::OS-UBI TERM + SB-ALS |
| | | | PRO::ZM-ALS::SB-UBI TERM + LB |
| 161 | DNA | PHP101206 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-T2S2C2-4 CTB + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 162 | DNA | PHP101207 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-T2S2C5 CTB + ZM-HSP17.7 |
| | | | PRO::MO-CRE EXON1::ST-LS1 INTRON2::MO-CRE |
| | | | EXON2::PINII TERM + LOXP + SB-UBI PRO::SB-UBI |
| | | | INTRON1::ZSGREEN1::OS-UBI TERM + SB-ALS |
| | | | PRO::ZM-ALS::SB-UBI TERM + LB |
| 163 | DNA | PHP101205 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-T1S2C9-2 CTB + ZM- |
| | | | HSP17.7 PRO::MO-CRE EXON1::ST-LS1 |
| | | | INTRON2::MO-CRE EXON2::PINII TERM + LOXP + |
| | | | SB-UBI PRO::SB-UBI INTRON1::ZSGREEN1::OS-UBI |
| | | | TERM + SB-ALS PRO::ZM-ALS::SB-UBI TERM + LB |
| 164 | DNA | PHP101094 | RB + LOXP + NOS PRO::ZM-WUS2::IN2 TERM + FMV |
| | | | ENHANCER + PCSV ENH + MMV ENH + UBI1ZM |
| | | | PRO::UBI1ZM 5UTR::UBI1ZM INTRON1::ZM- |
| | | | ODP2::OS-T28 TERM + ZM-T1S6C6 CTB + ZM-HSP17.7 |
| | | | PRO::MO-CRE EXON1::ST-LS1 INTRON2::MO-CRE |
| | | | EXON2::PINII TERM + LOXP + SB-UBI PRO::SB-UBI |
| | | | INTRON1::ZSGREEN1::OS-UBI TERM + SB-ALS |
| | | | PRO::ZM-ALS::SB-UBI TERM + LB |
| 165 | DNA | ZM-CTCF-1 CTB | A sequence with homology to CTCF-binding sites for |
| | | | CCCTC-binding factor which can function as a cross-talk |
| | | | blocker, blocking enhancer-promoter interaction. Found in |
| | | | Zea mays inbred line B73 on chromosome 4 located from |
| | | | 2660153 to 2660171. |
| 166 | DNA | 4X ZM-CTCF-1 | 4X multimerization of ZM-CTCF-1 CTB, a cross talk |
| | | (BZP1) | blocker from Zea mays with homology to CTCF-binding |
| | | | sites for CCCTC-binding factor. A unique 10 bp spacer is |
| | | | between each CTB. |
| 167 | DNA | ZM-CTCF-2 CTB | A sequence from Zea mays with homology to CTCF- |
| | | | binding sites for CCCTC-binding factor which can function |
| | | | as a cross-talk blocker, blocking enhancer-promoter |
| | | | interaction. |
| 168 | DNA | 4X ZM-CTCF-2 | 4X multimerization of CTCF-2 CTB, a cross talk blocker |
| | | (BZP1) | from Zea mays with homology to CTCF-binding sites for |
| | | | CCCTC-binding factor. A unique 10 bp spacer is between |
| | | | each CTB. |
| 169 | DNA | ZM-CTCF-3 CTB | A sequence from Zea mays with homology to CTCF- |
| | | | binding sites for which can function as a cross-talk blocker, |
| | | | blocking enhancer-promoter interaction.. |
| 170 | DNA | 4X ZM-CTCF-3 | 4X multimerization of ZM-CTCF-3 CTB, a cross talk |
| | | (BZP1) | blocker from Zea mays with homology to CTCF-binding |
| | | | sites for CCCTC-binding factor. A unique 10 bp spacer is |
| | | | between each CTB. |
| 171 | DNA | ZM-CTCF-4 CTB | A sequence from Zea mays with homology to CTCF- |
| | | | binding sites for CCCTC-binding factor which can function |
| | | | as a cross-talk blocker, blocking enhancer-promoter |
| | | | interaction. |
| 172 | DNA | 4X ZM-CTCF-4 | 4X multimerization of ZM-CTCF-4 CTB, a cross talk |
| | | (BZP1) | blocker from Zea mays with homology to CTCF-binding |
| | | | sites for CCCTC-binding factor. A unique 10 bp spacer is |
| | | | between each CTB. |
| 173 | DNA | ZM-CTCF-5 CTB | A sequence from Zea mays with homology to CTCF- |
| | | | binding sites for CCCTC-binding factor which can function |
| | | | as a cross-talk blocker, blocking enhancer-promoter |
| | | | interaction. |
| 174 | DNA | 4X ZM-CTCF-5 | 4X multimerization of ZM-CTCF-5 CTB, a cross talk |
| | | (BZP1) | blocker from Zea mays with homology to CTCF-binding |
| | | | sites for CCCTC-binding factor. A unique 10 bp spacer is |
| | | | between each CTB. |
| 175 | DNA | AT-DUF626 CTB | A sequence from Arabidopsis thaliana with homology to |
| | | | CTCF-binding sites for CCCTC-binding factor which can |
| | | | function as a cross-talk blocker, blocking enhancer- |
| | | | promoter interaction. |
| 176 | DNA | 4X AT-DUF626 | 4X multimerization of AT-DUF626 CTB, a cross talk |
| | | (BZP1) | blocker found in a protein with unknown function |
| | | | (DUF626) in Arabidopsis thaliana with homology to CTCF- |
| | | | binding sites for CCCTC-binding factor. A unique 10 bp |
| | | | spacer is between each CTB. |
| 177 | DNA | AT-PCD CTB | A sequence with homology to CTCF-binding sites for |
| | | | CCCTC-binding factor which can function as a cross-talk |
| | | | blocker, blocking enhancer-promoter interaction. Found in |
| | | | Arabidopsis thaliana polyketide cyclase/dehydrase gene on |
| | | | chromosome 1 from 5157810 to 5157828, reference |
| | | | sequence AT1G14950.1. |
| 178 | DNA | 4X AT-PCD (BZP1) | 4X multimerization of AT-PCD CTB, a cross talk blocker |
| | | | found in polyketide cyclase/dehydrase gene in Arabidopsis |
| | | | thaliana with homology to CTCF-binding sites for CCCTC- |
| | | | binding factor. A unique 10 bp spacer is between each CTB. |
| 179 | DNA | AT-DZFP CTB | A sequence from Arabidopsis thaliana with homology to |
| | | | CTCF-binding sites for CCCTC-binding factor which can |
| | | | function as a cross-talk blocker, blocking enhancer- |
| | | | promoter interaction. |
| 180 | DNA | 4X AT-DZFP (BZP1) | 4X multimerization of AT-DZFP CTB, a cross talk blocker |
| | | | found in a dof-type zinc finger DNA-binding protein in |
| | | | Arabidopsis thaliana with homology to CTCF-binding sites |
| | | | for CCCTC-binding factor. A unique 10 bp spacer is |
| | | | between each CTB. |
| 181 | DNA | AT-SAM CTB | A sequence from Arabidopsis thaliana with homology to |
| | | | CTCF-binding sites for CCCTC-binding factor which can |
| | | | function as a cross-talk blocker, blocking enhancer- |
| | | | promoter interaction. |
| 182 | DNA | 4X AT-SAM (BZP1) | 4X multimerization of AT-SAM CTB, a cross talk blocker |
| | | | found in S-adenosyl-L-methionine-dependent |
| | | | methyltransferase gene in Arabidopsis thaliana with |
| | | | homology to CTCF-binding sites for CCCTC-binding |
| | | | factor. A unique 10 bp spacer is between each CTB. |
| 183 | DNA | AT-DUF936 CTB | A sequence from Arabidopsis thaliana with homology to |
| | | | CTCF-binding sites for CCCTC-binding factor which can |
| | | | function as a cross-talk blocker, blocking enhancer- |
| | | | promoter interaction. |
| 184 | DNA | 4X AT-DUF936 | 4X multimerization of AT-DUF626 CTB, a cross talk |
| | | (BZP1) | blocker found in a protein with unknown function |
| | | | (DUF936) in Arabidopsis thaliana with homology to CTCF- |
| | | | binding sites for CCCTC-binding factor. A unique 10 bp |
| | | | spacer is between each CTB. |
| 185 | DNA | GM-CTCF-1 CTB | A sequence from Glycine max with homology to CTCF- |
| | | | binding sites for CCCTC-binding factor which can function |
| | | | as a cross-talk blocker, blocking enhancer-promoter |
| | | | interaction. |
| 186 | DNA | 4X GM-CTCF-1 | 4X multimerization of GM-CTCF-1 CTB, a cross talk |
| | | (BZP1) | blocker from Glycine max with homology to CTCF-binding |
| | | | sites for CCCTC-binding factor. A unique 10 bp spacer is |
| | | | between each CTB. |
| 187 | DNA | GM-CTCF-2 CTB | A sequence from Glycine max with homology to CTCF- |
| | | | binding sites for CCCTC-binding factor which can function |
| | | | as a cross-talk blocker, blocking enhancer-promoter |
| | | | interaction. |
| 188 | DNA | 4X GM-CTCF-2 | 4X multimerization of GM-CTCF-2 CTB, a cross talk |
| | | (BZP1) | blocker from Glycine max with homology to CTCF-binding |
| | | | sites for CCCTC-binding factor. A unique 10 bp spacer is |
| | | | between each CTB. |
| 189 | DNA | GM-MAT-1 CTB | A sequence from Glycine max with homology to CTCF- |
| | | | binding sites for CCCTC-binding factor which can function |
| | | | as a cross-talk blocker, blocking enhancer-promoter |
| | | | interaction. |
| 190 | DNA | 4X GM-MAT-1 | 4X multimerization of GM-MAT-1 CTB, a cross talk |
| | | (BZP1) | blocker found in mitochondrial ABC transporter gene in |
| | | | Glycine max with homology to CTCF-binding sites for |
| | | | CCCTC-binding factor. A unique 10 bp spacer is between |
| | | | each CTB. |
| 191 | DNA | GM-CTCF-3 CTB | A sequence from Glycine max with homology to CTCF- |
| | | | binding sites for CCCTC-binding factor which can function |
| | | | as a cross-talk blocker, blocking enhancer-promoter |
| | | | interaction. |
| 192 | DNA | 4X GM-CTCF-3 | 4X multimerization of GM-CTCF-3 CTB, a cross talk |
| | | (BZP1) | blocker from Glycine max with homology to CTCF-binding |
| | | | sites for CCCTC-binding factor. A unique 10 bp spacer is |
| | | | between each CTB. |
| 193 | DNA | GM-MAT-2 CTB | A sequence from Glycine max with homology to CTCF- |
| | | | binding sites for CCCTC-binding factor which can function |
| | | | as a cross-talk blocker, blocking enhancer-promoter |
| | | | interaction. |
| 194 | DNA | 4X GM-MAT-2 | 4X multimerization of GM-MAT-2 CTB, a cross talk |
| | | (BZP1) | blocker found in mitochondrial ABC transporter gene in |
| | | | Glycine max with homology to CTCF-binding sites for |
| | | | CCCTC-binding factor. A unique 10 bp spacer is between |
| | | | each CTB. |
| 195 | DNA | AT-CYT P450 CTB | A sequence with homology to BSwt of scs elements |
| | | | (Gaszner, et. al. 1999) found within the Cytochrome P450 |
| | | | gene in Arabidopsis thaliana to test as CTB |
| 196 | DNA | AT-ICGP CTB | A sequence with homology to BSwt of scs elements |
| | | | (Gaszner, et. al. 1999) found in Arabidopsis thaliana to test |
| | | | as CTB |
| 197 | DNA | AT-ITGP CTB | A sequence with homology to BSwt of scs elements |
| | | | (Gaszner, et. al. 1999) found in Arabidopsis thaliana to test |
| | | | as CTB |
| 198 | DNA | AT-PHY A CTB | A sequence with homology to BSwt of scs elements |
| | | | (Gaszner, et. al. 1999) found in Arabidopsis thaliana to test |
| | | | as CTB |
| 199 | DNA | AT-RING CTB | A sequence with homology to BSwt of scs elements |
| | | | (Gaszner, et. al. 1999) found in Arabidopsis thaliana to test |
| | | | as CTB |
| 200 | DNA | ZM-IDGP CTB | A sequence with homology to BSwt of scs elements |
| | | | (Gaszner, et. al. 1999) found in Zea mays to test as CTB |
| 201 | DNA | ZM-ICGP CTB | A sequence with homology to BSwt of scs elements |
| | | | (Gaszner, et. al. 1999) found in Zea mays to test as CTB |
| 202 | DNA | ZM-IDGP-2 CTB | A sequence with homology to BSwt of scs elements |
| | | | (Gaszner, et. al. 1999) found in Zea mays to test as CTB |
| 203 | DNA | ZM-IDGP-3 CTB | A sequence with homology to BSwt of scs elements |
| | | | (Gaszner, et. al. 1999) found in Zea mays to test as CTB |
| 204 | DNA | ZM-IDGP-4 CTB | A sequence with homology to BSwt of scs elements |
| | | | (Gaszner, et. al. 1999) found in Zea mays to test as CTB |
| 205 | DNA | GM-CYT P450 CTB | A sequence with homology to BSwt of scs elements |
| | | | (Gaszner, et. al. 1999) found in Glycine max to test as CTB |
| 206 | DNA | GM-CYT P450-2 CTB | A sequence with homology to BSwt of scs elements |
| | | | (Gaszner, et. al. 1999) found in Glycine max to test as CTB |
| 207 | DNA | GM-IDGP CTB (REV) | A sequence with homology to Scs binding protein (SBP) |
| | | | sequence from Glycine max to test as CTB |
| 208 | DNA | GM-IDGP CTB | A sequence with homology to BSwt of scs elements |
| | | | (Gaszner, et. al. 1999) found in Glycine max to test as CTB |
| 209 | DNA | GM-CYT P450-3 CTB | A sequence with homology to BSwt of scs elements |
| | | | (Gaszner, et. al. 1999) found in Glycine max to test as CTB |
| 210 | DNA | ZM-TBS1 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 211 | DNA | ZM-TBS2 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 212 | DNA | ZM-TBS3 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 213 | DNA | ZM-TBS4 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 214 | DNA | ZM-TBS5 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 215 | DNA | ZM-TBS6 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 216 | DNA | ZM-TBS7 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 217 | DNA | ZM-TBS8 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 218 | DNA | ZM-TBS9 CTB | Orthologous sequence in Zea mays to Petunia hybrida |
| | | | transformation booster sequence (TBS), which blocks |
| | | | enhancer-promoter activity (Hily, Singer et al, 2009) |
| | | | making it a cross-talk blocker. |
| 219 | DNA | PH-TBS CTB (TR1) | Truncated version of PH-TBS CTB, a transformation |
| | | | booster sequence (TBS) from Petunia hybrida which blocks |
| | | | enhancer-promoter activity (Hily, Singer et al, 2009), |
| | | | making it a cross-talk blocker. |
| 220 | DNA | SL-TBS CTB | Orthologous sequence in Solanum lycopersicum to Petunia |
| | | | hybrida transformation booster sequence (TBS), which |
| | | | blocks enhancer-promoter activity (Hily, Singer et al, 2009) |
| | | | making it a cross-talk blocker. |
| 221 | DNA | OS-DHS1 (MOD1) | Intergenic region from Oryza sativa, with high DNase |
| | | | hypersensitivity, genomic location is |
| | | | Chr7: 16144038 . . . 16144473. |
| 222 | DNA | OS-DHS2 (MOD1) | Intergenic region from Oryza sativa, with DNase |
| | | | hypersensitivity, genomic location is |
| | | | Chr8: 21048284 . . . 21048731. |
| 223 | DNA | OS-DHS3 (MOD1) | Intergenic region from Oryza sativa, with DNase |
| | | | hypersensitivity, genomic location is |
| | | | Chr2: 28170208 . . . 28170687. |
| 224 | DNA | OS-DHS4 (MOD1) | Intergenic region from Oryza sativa, with DNase |
| | | | hypersensitivity, genomic location is |
| | | | Chr4: 33443937 . . . 33444440. |
| 225 | DNA | OS-DHS5 (MOD1) | Intergenic region from Oryza sativa, with DNase |
| | | | hypersensitivity, genomic location is |
| | | | Chr5: 28025699 . . . 28026132. |
| 226 | DNA | OS-DHS6 (MOD1) | Intergenic region from Oryza sativa, with DNase |
| | | | hypersensitivity, genomic location is |
| | | | Chr6: 16292319 . . . 16292667. |
| 227 | DNA | OS-DHS7 (MOD1) | Intergenic region from Oryza sativa, with DNase |
| | | | hypersensitivity, genomic location is |
| | | | Chr8: 25636379 . . . 25636878. |
| 228 | DNA | OS-DHS8 (MOD1) | Intergenic region from Oryza sativa, with DNase |
| | | | hypersensitivity, genomic location is |
| | | | Chr8: 25621000 . . . 25621379. |
| 229 | DNA | OS-DHS9 (MOD1) | Intergenic region from Oryza sativa, with DNase |
| | | | hypersensitivity, genomic location is |
| | | | Chr9: 17275575 . . . 17276285. |
| 230 | DNA | OS-DHS10 (MOD1) | Intergenic region from Oryza sativa, with DNase |
| | | | hypersensitivity, genomic location is |
| | | | Chr10: 22658189 . . . 22658479. |
| 231 | DNA | OS-DHS15 (MOD1) | Intergenic region from Oryza sativa, with DNase |
| | | | hypersensitivity, genomic location is |
| | | | Chr8: 21042139 . . . 21042670. |
| 232 | DNA | OS-DHS18 (MOD1) | Intergenic region from Oryza sativa, with high DNase |
| | | | hypersensitivity, genomic location is |
| | | | Chr7: 22384243 . . . 22384593. |
| 233 | DNA | OS-DHSC2 ENH1 | A 31 bp sequence with in the DHS co-ordinates |
| | | (TR1) | Chr2: 31783851 . . . 31785303. |
| 234 | DNA | OS-DHSC5 ENH1 | A 31 bp sequence with in the DHS co-ordinates |
| | | (TR1) | Chr5: 22452532 . . . 22453378. |
| 235 | DNA | BD-DHS3 | Intergenic region from Brachypodium distachyon, with |
| | | | DNase hypersensitivity, genomic location is |
| | | | Chr3: 20961596 . . . 20961857. |
| 236 | DNA | BD-DHS6 (MOD1) | Intergenic region from Brachypodium distachyon, with |
| | | | DNase hypersensitivity, genomic location is |
| | | | Chr1: 19108672 . . . 19109075. |
| 237 | DNA | BD-DHS7 (MOD1) | Intergenic region from Brachypodium distachyon, with |
| | | | DNase hypersensitivity, genomic location is |
| | | | Chr3: 47221260 . . . 47221579. |
| 238 | DNA | BD-DHS8 | Intergenic region from Brachypodium distachyon, with |
| | | | DNase hypersensitivity, genomic location is |
| | | | Chr1: 58189954 . . . 58190135. |
| 239 | DNA | BD-DHS11 (MOD1) | Intergenic region from Brachypodium distachyon, with |
| | | | DNase hypersensitivity, genomic location is |
| | | | Chr3: 38853080 . . . 38853719. |
| 240 | DNA | BD-DHS12 (MOD1) | Intergenic region from Brachypodium distachyon, with |
| | | | DNase hypersensitivity, genomic location is |
| | | | Chr1: 20083313 . . . 20083880. |
| 241 | DNA | BD-DHS16 (MOD1) | Intergenic region from Brachypodium distachyon, with |
| | | | DNase hypersensitivity, genomic location is |
| | | | Chr1: 35765660 . . . 35768479. |
| 242 | DNA | BD-DHS18 (MOD1) | Intergenic region from Brachypodium distachyon, with |
| | | | DNase hypersensitivity, genomic location is |
| | | | Chr2: 14391681 . . . 14392228. |
| 243 | DNA | SI-LTP2-2 UAR1 | Upstream Activation Region 1 of Setaria italica |
| | | | Bifunctional inhibitor/lipid-transfer protein/seed storage 2S |
| | | | albumin-like protein/Probable lipid transfer (LTP_2) |
| | | | promoter; reference sequence Seita.9G570900; edited to |
| | | | remove unwanted ORFs and RE sites; from −994 to −55 (no |
| | | | TATA) |
| 244 | DNA | ZM-NACI UAR1 | Upstream Activation Region 1 of Zea mays NaCl stress |
| | | | protein 1 (nac1) promoter; reference sequence |
| | | | dpzm10g022630.1.1; GRMZM2G015605_T01; from −1025 |
| | | | to −52 (no TATA); edited to remove unwanted ORF. |
| 245 | DNA | BD-LHCB2 (MOD1) | Upstream Activation Region 1 of Brachypodium distachyon |
| | | UAR1 | light-harvesting complex II chlorophyll a/b binding protein |
| | | | 2 (LHCB2); reference sequence Bradi1g15290.1; edited to |
| | | | remove unwanted RE sites and ORF; from −1017 to −55 (no |
| | | | TATA) |
| 246 | DNA | ZM-THAUMATIN | Upstream Activation Region 1 of Zea mays Thaumatin, |
| | | UAR1 | pathogenesis-related (similar to prp5) promoter; reference |
| | | | sequence dpzm01g055930.1.1; GRMZM2G402631_T01; |
| | | | from −1010 to −54 (no TATA.) |
| 247 | DNA | BD-LTP2 UAR1 | Upstream activation region 1 of the Brachypodium |
| | | | distaychon tonoplast intrinsic protein/aquaporin transporter |
| | | | promoter from −1006 to −53 (no TATA), reference sequence |
| | | | Bradi2g17550.1 edited to remove ORF. |
| 248 | DNA | BD-GST UAR1 | Upstream activation region 1 of Brachypodium distaychon |
| | | | Glutathione S-transferase promoter; reference sequence |
| | | | Bradi2g13110.1; from −1006 to −57 (no TATA); edited to |
| | | | remove RE site and ORF. BglII sites not removed. |
| 249 | DNA | ZM-PLTP UAR1-V1 | Upstream activation region 1 from the zea mays |
| | | | phospholipid transfer protein promoter, reference sequence |
| | | | GRMZM2G101958 (historical reference |
| | | | dpzm10g001940.1.1). From −952 to −56 (no TATA). edited |
| | | | to remove unwanted RE site |
| 250 | DNA | SB-DUF3054 UAR1 | Upstream Activation Region 1 of Sorghum bicolor protein |
| | | | of unknown function (DUF3054) promoter; from −1120 |
| | | | to −56 (no TATA); reference sequence Sobic.001G104800; |
| | | | edited to removed unwanted RE sites. |
| 251 | DNA | STV6 | 3 kb coding sequence amplified from cDNA of Arobidopsis |
| | | | thaliana AT2G26080.1 gene as CTB test |
| 252 | DNA | STV7 | 1.7 Kb coding sequence amplified from cDNA of |
| | | | Arobidopsis thaliana AT2G26080.1 gene as CTB test |
| 253 | DNA | STV8 | Intergenic region and 3¹ UTR from Convergent gene pair as |
| | | | CTB |
| 254 | DNA | STV9 | 4x terminator as CTB |
| 255 | DNA | STV10 | 4x terminator in reverse as CTB |
| 256 | DNA | STV11 | Intergenic region and 3¹ UTR from Convergent gene pair as |
| | | | CTB |
| 257 | DNA | STV12 | 4x terminator as CTB |
| 258 | DNA | STV13 | 4x terminator in reverse as CTB test |
| 259 | DNA | STV14 | Intergenic region and 3¹ UTR from Convergent gene pair as |
| | | | CTB |
| 260 | DNA | STV15 | 4x terminator as CTB |
| 261 | DNA | STV16 | 4x terminator in reverse as CTB test |
| 262 | DNA | STV17 | 1 Kb coding sequence as CTB test |
| 263 | DNA | STV18 | Poly A signal sequences from 5 Terminators as CTB |
| 264 | DNA | STV19 | Poly A signal sequences from 5 Terminators in reverse as |
| | | | CTB test |
| 265 | DNA | STV20 | Poly A signal sequences from 5 Terminators as CTB |
| 266 | DNA | STV21 | Poly A signal sequences from 5 Terminators in reverse as |
| | | | CTB test |
| 267 | DNA | OVERLAPPING DCM | String of 25 restriction endonuclease sites each blocked by |
| | | BLOCK | overlapping Dcm methylation sequence CCWGG (where W |
| | | | is A or T). From 5 prime to 3 prime those sites are Acc65I, |
| | | | AlwNI, ApaI, AvaII, BanI, BsaI, BsaHI, BslI, BssKI, |
| | | | BstXI, EaeI, EcoO109I, MscI, NlaIV, PflMI, PpuMI, |
| | | | PspGI, PspOMI, Sau96I, ScrFI, SexAI, SfiI(x2), SfoI, StuI. |

That which is claimed is:

1. A recombinant polynucleotide construct comprising:
at least two cassettes, wherein each cassette comprises a promoter operably linked to a heterologous gene; and
at least one cross-talk blocking element;
wherein the cross-talk blocking element comprises a polynucleotide sharing at least 80% identity with at least 100 contiguous nucleotides of any one of SEQ ID NO: 1-267.

2. A recombinant polynucleotide construct comprising:
at least two cassettes, wherein each cassette comprises a promoter operably linked to a heterologous gene; and
at least one cross-talk blocking element;
wherein the cross-talk blocking element comprises any one or more motif(s) as described in Table 13.

3. A recombinant polynucleotide construct comprising:
at least two cassettes, wherein each cassette comprises a promoter operably linked to a heterologous gene; and
at least one cross-talk blocking element;
wherein the cross-talk blocking element is a Type I or Type II cross-talk blocking element.

4. The recombinant polynucleotide construct of claim 1, wherein the cross-talk blocking element is adjacent to one of the at least two cassettes.

5. The recombinant polynucleotide construct of claim 1, wherein the cross-talk blocking element is adjacent to at least two of the at least two cassettes.

6. The recombinant polynucleotide construct of claim 1, wherein at least one of the promoters of the at least two cassettes is constitutive.

7. The recombinant polynucleotide construct of claim 1, wherein at least one of the promoters of the at least two cassettes is tissue-specific or developmental stage-specific.

8. A plant cell comprising the recombinant polynucleotide construct of claim 1.

9. The plant cell of claim 8, selected from the group consisting of: maize, soybean, Arabidopsis, canola, wheat, rice, tobacco, cotton, alfalfa, sorghum, sunflower, or safflower.

10. A transgenic plant comprising the recombinant polynucleotide construct of claim 1 in at least one cell.

11. A method of modulating the expression of at least one transgene in a plant cell, the method comprising:
introducing into the plant cell the recombinant construct of claim 1,
incubating the cell under conditions that allow the expression of the transgene, and
assessing the expression of said transgene;
wherein the expression of said at least one transgene is modulated compared to that of a control plant comprising the transgene but lacking the cross-talk blocker.
